SEQUENCE LISTING (9 sequences)

SEQ ID NO: 1 - DNA, 26 bp, Artificial
  primer
ctcgagatggcaactctaaaggatca

SEQ ID NO: 2 - DNA, 28 bp, Artificial
  primer
gcggccgcttaaaattgcagctcctttt

SEQ ID NO: 3 - DNA, 999 bp, Homo sapiens
atggcaactctaaaggatcagctgatttataatcttctaaaggaagaacagaccccccagaataagattacagttgttggggttggtgctgttggcatggcctgtgccatcagtatcttaatgaaggacttggcagatgaacttgctcttgttgatgtcatcgaagacaaattgaagggagagatgatggatctccaacatggcagccttttccttagaacaccaaagattgtctctggcaaagactataatgtaactgcaaactccaagctggtcattatcacggctggggcacgtcagcaagagggagaaagccgtcttaatttggtccagcgtaacgtgaacatatttaaattcatcattcctaatgttgtaaaatacagcccgaactgcaagttgcttattgtttcaaatccagtggatatcttgacctacgtggcttggaagataagtggttttcccaaaaaccgtgttattggaagtggttgcaatctggattcagcccgattccgttacctgatgggggaaaggctgggagttcacccattaagctgtcatgggtgggtccttggggaacatggagattccagtgtgcctgtatggagtggaatgaatgttgctggtgtctctctgaagactctgcacccagatttagggactgataaagataaggaacagtggaaagaggttcacaagcaggtggttgagagtgcttatgaggtgatcaaactcaaaggctacacatcctgggctattggactctctgtagcagatttggcagagagtataatgaagaatcttaggcgggtgcacccagtttccaccatgattaagggtctttacggaataaaggatgatgtcttccttagtgttccttgcattttgggacagaatggaatctcagaccttgtgaaggtgactctgacttctgaggaagaggcccgtttgaagaagagtgcagatacactttgggggatccaaaaggagctgcaattttaa

SEQ ID NO: 4 - DNA, 80 bp, Artificial
  primer
tctcaattattattttctactcataacctcacgcaaaataacacagtcaaatcaatcaaaatggcaactctaaaggatca

SEQ ID NO: 5 - DNA, 20 bp, Artificial
  primer
aggcgtatcacgaggccctt

SEQ ID NO: 6 - DNA, 60 bp, Artificial
  primer
gaattaattcttgaagacgaaagggcctcgtgatacgcctagattgtactgagagtgcac

SEQ ID NO: 7 - DNA, 80 bp, Artificial
  primer
tatttttcgttacataaaaatgcttataaaactttaactaataattagagattaaatcgcctgtgcggtatttcacaccg

SEQ ID NO: 8 - DNA, 25 bp, Artificial
  primer
caaatatcgtttgaatatttttccg

SEQ ID NO: 9 - DNA, 20 bp, Artificial
  primer
aatccagattgcaaccactt

(19) United States
(12) Patent Application Publication
(10) Pub. No.: US 2013/0149745 A1
(43) Pub. Date: Jun. 13, 2013

(54) METHOD FOR PRODUCING CHEMICALS BY CONTINUOUS FERMENTATION

(71) Applicants: Jihoon Cheon, Otsu (JP); Shin-ichi Minegishi, Urayasu (JP); Makoto Nishida, Otsu (JP); Norihiro Takeuchi, Otsu (JP)

(72) Inventors: Jihoon Cheon, Otsu (JP); Shin-ichi Minegishi, Urayasu (JP); Makoto Nishida, Otsu (JP); Norihiro Takeuchi, Otsu (JP)

(73) Assignee: TORAY INDUSTRIES, INC., Tokyo (JP)

(21) Appl. No.: 13/575,039

(22) PCT Filed: Jan. 24, 2011
(86) PCT No.: PCT/JP2011/051213

§ 371 Date: Sep. 25, 2012

(30) Foreign Application Priority Data
Jan. 28, 2010 (JP) 2010-016330

(51) Int. Cl. C12P 7/56; C12P 7/14; C12P 7/46

(52) U.S. Cl. CPC C12P 7/56; C12P 7/46; C12P 7/14
USPC 435/91.1; 435/145; 435/162; 435/139

(57) ABSTRACT

A method produces a chemical by fermentation including: filtering a liquid containing a feedstock, the chemical, and bacterial, microbial or cultured cells through a membrane to recover the chemical from the filtrate; retaining or refluxing unfiltered liquid in the liquid; and adding the feedstock to the liquid, wherein the membrane is a porous hollow-fiber membrane including a polyvinylidene fluoride resin, the porous hollow-fiber membrane having an average pore size of 0.001 μm to 10.0 μm, pure water permeability coefficient at 50 kPa at 25° C. of 0.5 m³/m²/hr to 15 m³/m²/hr, breaking strength of 5 MPa to 20 MPa, elongation at break of 30% to 200%, crimping degree of 1.3 to 2.5, porosity of not less than 40%, and critical surface tension of 45 mN/m to 75 mN/m.

### RELATED APPLICATIONS

This is a §371 of International Application No. PCT/JP2011/051213, with an inter-national filing date of Jan. 24, 2011 (WO 2011/093241 A1, published Aug. 4, 2011), which is based on Japanese Patent Application No. 2010-016330, filed Jan. 28, 2010, the subject matter of which is incorporated by reference.

### TECHNICAL FIELD

This disclosure relates to a method for producing a chemical by continuous fermentation.

### BACKGROUND

Fermentation methods, which are accompanied by culture of bacterial, microbial or cultured cells, are largely classified into (1) batch fermentation and fed-batch or semi-batch fermentation; and (2) continuous fermentation. Batch fermentation and fed-batch or semi-batch fermentation have advantages in that they need only simple equipment, culturing can be finished in a short time and, in cases of chemical product fermentation by pure microorganism culture, contamination with microorganisms other than the cultured microorganism required for the culture is less likely to occur. However, the product concentration in the fermentation liquid increases with time, and this causes inhibition of the product, an increase in the osmotic pressure and the like, leading to a decrease in the productivity and the yield. Therefore, it is difficult to stably maintain high yield and high productivity for a long time.

In continuous fermentation, high yield and high productivity can be maintained for a longer time compared to the above cases of batch fermentation and fed-batch or semi-batch fermentation since accumulation of the substance of interest in the fermenter can be avoided. Conventional continuous culture is a culture method wherein a fresh medium is supplied to a fermenter at a constant rate, while the same amount of fermentation liquid is discharged to the outside of the fermenter, by which the amount of liquid in the fermenter is kept constant. In batch fermentation, culturing is terminated upon consumption of a substrate which was initially at a certain concentration. However, theoretically, in continuous fermentation, culturing can be infinitely continued. That is, theoretically, fermentation can be infinitely continued.

However, since in conventional continuous culturing, microorganisms are discharged together with the fermentation liquid to the outside of the fermenter, it is difficult to keep the concentration of the microorganisms in the fermenter. Thus, it is possible when fermentative production is carried out to increase the fermentative production efficiency per fermentation volume if the concentration of the microorganisms with which the fermentation is performed can be kept high. To achieve this, the microorganisms need to be retained or refluxed in the fermenter.

Examples of the method for retaining or refluxing the microorganisms in the fermenter include a method wherein discharged fermentation liquid is centrifuged for solid-liquid separation and microorganisms as a precipitate are returned into the fermenter, and a method wherein microorganisms as solids are separated by filtration and only the fermentation liquid supernatant is discharged to the outside of the fermenter. However, the method by centrifugation is unrealistic because of requirement of a high power cost. Application of the method by filtration has been mostly limited to laboratory studies since this method requires high pressure for the filtration described above. As an example of this method, a continuous fermentation method for L-glutamic acid and L-lysine has been disclosed (Toshihiko Hirao et al., Appl. Microbiol. Biotechnol., 32, 269-273 (1989)). However, in these examples, although continuous fermentation is performed by continuous addition of a fermentation feedstock to a fermenter, fermentation liquid containing bacterial, microbial or cultured cells is withdrawn so that the bacterial, microbial or cultured cells in the fermentation liquid are removed and diluted at the same time, resulting in decrease in the microorganism concentration in the fermenter and hence in only limited improvement of the production efficiency.

Therefore, in continuous fermentation, a method has been proposed wherein bacterial, microbial or cultured cells are separated/filtered with a separation membrane to recover a chemical from the filtrate, while the separated bacterial, microbial or cultured cells are retained or refluxed in the fermentation liquid, by which the concentration of the microorganisms or cells in the fermentation liquid is kept high. For example, for continuous fermentation apparatuses using ceramic membranes, techniques related to membrane separation continuous fermentation have been disclosed (JP 5-95778 A, JP 62-138184 A and JP 10-174594 A). In these techniques, superiority of membrane separation continuous fermentation over existing continuous fermentation was shown since the concentration of microorganisms or cultured cells can be kept high by membrane separation. However, in the disclosed techniques, there are problems such as decrease in the filtration flow rate and the filtration efficiency due to clogging of the ceramic membrane so that back-pressure washing and the like are carried out for prevention of clogging.

On the other hand, in recent years, a method for producing succinic acid using a separation membrane has also been disclosed (JP 2005-333886 A). This technique uses not only a ceramic membrane as described above but also an organic membrane, expanding the range and the types of the membranes applicable to the continuous fermentation technology. However, in the disclosed technique, a high filtration pressure (about 200 kPa) and a high membrane surface linear velocity (2 m/s) are employed in membrane separation. Employment of high filtration pressure and high membrane surface linear velocity is not appropriate in continuous fermentation wherein bacterial, microbial or cultured cells are continuously returned into the fermenter, not only because of the high cost but also because of the facts that the bacterial, microbial or cultured cells may be physically damaged during filtration by the high pressure/high velocity and that the pressure loss is likely to occur, making maintenance of the operating conditions difficult.

On the other hand, separation membranes have been more and more applied to, in addition to the above-described field of fermentation, the field of tap water in which river water and the like are clarified to produce drinking water and industrial water, and the field of sewage in which sewage (sewage secondary treatment effluent) is clarified and purified. For wide use of the membranes in such fields, treatment that prevents contamination (clogging) with organic substances and the like as much as possible is necessary. As materials for the membranes, various materials such as cellulose materials, polyacrylonitrile materials and polyolefin materials are used. Among these, polyvinylidene fluoride is suitable and hopeful as a material for an aqueous filtration membrane since it has high strength and high heat resistance, and has high water resistance due to its hydrophobic skeleton.

As a production method for a polyvinylidene fluoride membrane, U.S. Pat. No. 5,022,990 B proposes a method for producing a hollow-fiber membrane, wherein polyvinylidene fluoride, an organic liquid and an inorganic fine powder are melt-kneaded and subjected to microphase separation by cooling, followed by extraction of the organic liquid and the inorganic fine powder. Further, WO 91/17204 discloses a method for producing a hollow-fiber membrane composed of polyvinylidene fluoride and a solvent system. However, it is generally known that, in cases where highly polluted raw water is filtered through these membranes, deposit remaining unfiltered on the membrane surface or inside the membrane causes additional filtration resistance, leading to decreased filtration performance. Therefore, flushing in which deposit is peeled off with rapid water stream, air scrubbing, in which deposit is peeled off by blowing air bubbles onto the filter, backwashing, in which the direction of filtration is reversed to wash the filter and the like are employed, during which the filtration operation is interrupted. Further, the filtration performance is kept high also by periodically washing the filter with a chemical. Although the washing effects of flushing and air scrubbing are high, these steps impose high loads on the membrane, so that the membrane is likely to be broken. Further, since, in the case of a conventional membrane, influence of accumulation of contaminants (clogging) on the membrane with time is still large even if these means are employed, satisfactory permeability cannot necessarily be obtained, which has been problematic.

On the other hand, recently, a method has been proposed wherein a separation membrane is applied to a fermentation method, which is a method for producing a substance accompanied by culturing microorganisms, to carry out continuous fermentation, thereby allowing accumulation of bacterial, microbial or cultured cells to increase the production rate (JP 2008-237213 A). In that technique, a fermenter is first provided, and a membrane separation vessel containing a flat membrane and a hollow-fiber membrane is provided. Using a pump, the fermentation liquid is fed from the fermenter to the membrane separation vessel, and filtration is controlled using a hydraulic head difference controlling device provided separately from the membrane unit in the membrane separation vessel. However, in that method, there are problems in placement and maintenance of the equipments since, for example, the two vessels and the control unit are provided. Hence, a large installation space is required, and exchanging of the separation membrane needs to be carried out after blocking the membrane separation vessel from the fermenter to stop operation of the vessel, resulting in decrease in the production rate. Further, the proposal to place a separation membrane in the fermenter means that the fermenter needs to be stopped upon exchanging the separation membrane, so that the production may need to be stopped due to a problem that arose from the separation membrane. Further, in cases where the operation of continuous fermentation is carried out using the separation membrane disclosed in JP '213, a stable continuous operation cannot be achieved for a long time, which is problematic.

On the other hand, recently, research has been carried out in an attempt to improve the production rate by fermentation to which a separation membrane is applied. Guo-qian Xu et al., Process Biochemistry, 41, 2458-2463 (2006) suggests that the production rate may be improved by performing lactic acid fermentation using Lactobacillus paracasei and filtering the fermentation liquid using a membrane composed of polyvinylidene fluoride. However, since washing with sodium hypochlorite is carried out to avoid an increase in the transmembrane pressure difference due to membrane clogging, frequent washing during long-term filtering operation may cause problems such as adverse effects on fermentation due to decomposition of fermentative microorganisms by hypochlorous acid or accumulation of sodium, and occurrence of leakage due to deterioration of the hollow-fiber membrane. Further, in spite of the fact that the membrane filtration operation was accompanied by washing with a chemical, the operation could be continued for a maximum of only 150 hours. With such a short operation time, continuous fermentation is hardly advantageous over batch operation even in view of the cost, and the method cannot therefore be easily applied to practical fermentation and production. Therefore, improvement in the technique has been demanded.

It could therefore be helpful to provide a method for producing a chemical by continuous fermentation, wherein high productivity can be stably maintained for a long time by a simple operation method.

### SUMMARY

We thus provide:
(1) A method for producing a chemical by continuous fermentation comprising:
   filtering a fermentation liquid containing a fermentation feedstock, the chemical, and bacterial, microbial or cultured cells through a separation membrane to recover the chemical from the filtrate;
   retaining or refluxing unfiltered liquid in the fermentation liquid; and
   adding the fermentation feedstock to the fermentation liquid;
   wherein the separation membrane is a porous hollow-fiber membrane composed of a polyvinylidene fluoride resin,
   the porous hollow-fiber membrane having an average pore size of not less than 0.001 μm and not more than 10.0 μm, pure water permeability coefficient at 50 kPa at 25° C. of not less than 0.5 m³/m²/hr and not more than 15 m³/m²/hr, breaking strength of not less than 5 MPa and not more than 20 MPa, elongation at break of not less than 30% and less than 200%, crimping degree of not less than 1.3 and not more than 2.5, porosity of not less than 40%, and critical surface tension of not less than 45 mN/m and not more than 75 mN/m.
(2) The method for producing a chemical by continuous fermentation according to the above (1), wherein the surface of the porous hollow-fiber membrane is coated with an ethylene-vinyl alcohol copolymer.
(3) The method for producing a chemical by continuous fermentation according to the above (1) or (2), wherein the porous hollow-fiber membrane is prepared by impregnating a porous hollow-fiber membrane composed of a polyvinylidene fluoride resin with an ethylene-vinyl alcohol copolymer solution comprising an ethylene-vinyl alcohol copolymer and a solvent which is inert to polyvinylidene fluoride and dissolves the ethylene-vinyl alcohol copolymer, followed by drying treatment.
(4) The method for producing a chemical by continuous fermentation according to any one of the above (1) to (3), wherein the fermentation feedstock comprises a saccharide.
(5) The method for producing a chemical by continuous fermentation according to any one of the above (1) to (4), wherein the chemical is an organic acid, alcohol or nucleic acid.

Since continuous fermentation can be stably carried out for a long time with high productivity with our methods, chemicals as fermentation products can be stably produced at low cost generally in the fermentation industry.

### BRIEF DESCRIPTION OF THE DRAWINGS

FIG. 1 is a schematic diagram for explaining an example of our membrane-separation-type continuous fermentation apparatus.

FIG. 2 is a schematic diagram for explaining an example of our separation membrane module.

FIG. 3 is a diagram showing a physical map of pTRS11, a yeast expression vector.

### DESCRIPTION OF SYMBOLS

1. Fermenter
2. Separation membrane module
3. Temperature controller
4. Gas supplying apparatus
5. Stirrer
6. Differential pressure gauge
7. Medium-supplying pump
8. pH adjustment solution supplying pump
9. pH sensor/controlling device
10. Fermentation liquid circulating pump
11. Level sensor
12. Filtration pump
21. Lower resin sealing layer
22. Separation membrane bundle
23. Upper resin sealing layer
24. Liquid collection pipe

### DETAILED DESCRIPTION

We provide a method for producing a chemical by continuous fermentation, the method comprising: filtering a fermentation liquid comprising a fermentation feedstock, the chemical, and bacterial, microbial or cultured cells through a separation membrane to recover the chemical from the filtrate; retaining or refluxing unfiltered liquid in the fermentation liquid; and adding the fermentation feedstock to the fermentation liquid; which method uses as the separation membrane a porous hollow-fiber membrane composed of a polyvinylidene fluoride resin, the porous hollow-fiber membrane having an average pore size of not less than 0.001 μm and not more than 10.0 μm, pure water permeability coefficient at 50 kPa at 25° C. of not less than 0.5 m³/m²/hr and not more than 15 m³/m²/hr, breaking strength of not less than 5 MPa and not more than 20 MPa, elongation at break of not less than 30% and less than 200%, crimping degree of not less than 1.3 and not more than 2.5, porosity of not less than 40%, and critical surface tension of not less than 45 mN/m and not more than 75 mN/m.

The porous membrane is in the form of a hollow-fiber membrane. Hollow-fiber membranes are advantageous when they are prepared into the forms in which they are actually used in filtration (modules) since the area of the filling membrane per unit volume can be made larger compared to flat membranes and sheet-shaped membranes, allowing higher filtration performance per unit volume.

Since polyvinylidene fluoride has high strength and high heat resistance, and has high water resistance due to its hydrophobic skeleton, it is suitable as a material for our method. Examples of the polyvinylidene fluoride include vinylidene fluoride homopolymers and vinylidene fluoride copolymers. Examples of the vinylidene fluoride copolymers include copolymers of vinylidene fluoride with one or more of monomers selected from the group consisting of tetrafluoroethylene, hexafluoropropylene, chlorotrifluoroethylene and ethylene. Vinylidene fluoride homopolymers are preferably used. These polymers may be used either individually or as a mixture of 2 or more thereof.

The weight average molecular weight (Mw) of the polyvinylidene fluoride is preferably not less than 100,000 and less than 1,000,000. In cases where Mw of the polyvinylidene fluoride is less than 100,000, the obtained hollow-fiber membrane is impractical since it shows less elongation and is fragile, while in cases where Mw is not less than 1,000,000, the fluidity during melting is low, leading to worse moldability.

The hollow-fiber membrane is preferably produced from a mixture composed of three components, that is, polyvinylidene fluoride, an organic liquid and an inorganic fine powder. The inorganic fine powder has a function as a carrier to retain the organic liquid, and also has a function as nuclei for microphase separation. That is, the inorganic fine powder prevents detachment of the organic liquid during melt-kneading and molding of the mixture, thereby making the molding easy, and has a function, as nuclei for microphase separation to allow high-level microdispersion of the organic liquid and to prevent aggregation of the organic liquid. As the inorganic fine powder, hydrophobic silica is preferably used. Since hydrophobic silica is not likely to cause aggregation, it is finely microdispersed during melt-kneading and molding, to form a uniform three-dimensional network structure as a result. The hydrophobic silica herein means silica hydrophobized by reacting silanol groups on the surface of the silica with an organosilicon compound such as dimethylsilane or dimethyldichlorosilane, thereby replacing the surface of the silica with methyl groups or the like.

The organic liquid means a liquid having a boiling point of not less than 150° C. The organic liquid is extracted from the hollow-fiber membrane and makes the resulting hollow-fiber membrane porous. Preferably, the organic liquid is not compatible with polyvinylidene fluoride at low temperature (normal temperature), but is compatible with polyvinylidene fluoride during melt-molding (at high temperature).

A hollow-fiber membrane which may be used as the separation membrane of may be prepared by a production method in which a mixture composed of polyvinylidene fluoride and an organic liquid, or a mixture composed of polyvinylidene fluoride, an organic liquid and an inorganic fine powder, is melt-kneaded and extruded to mold a hollow-fiber membrane, followed by extracting the organic liquid, or the organic liquid and the inorganic fine powder. The production process of the hollow-fiber membrane preferably comprises a step wherein the hollow-fiber membrane before completion of the extraction or the hollow-fiber membrane after completion of the extraction is stretched in the longitudinal direction of the fiber and then contracted in the longitudinal direction of the fiber. The porous hollow-fiber membrane may be produced by, for example, impregnating a polyvinylidene fluoride resin hollow-fiber membrane obtained by the above method with an ethylene-vinyl alcohol copolymer solution comprising an ethylene-vinyl alcohol copolymer and a solvent which is inert to polyvinylidene fluoride and dissolves the ethylene-vinyl alcohol copolymer, followed by drying treatment.

Further, the hollow-fiber membrane is preferably crimped during the contraction step. By this, a highly crimped hollow-fiber membrane can be obtained with neither collapse nor damage. In general, hollow-fiber membranes have straight tubular shapes without bends. Therefore, it is highly possible that, when they are bundled to prepare a filtration module, gaps between the hollow fibers cannot be secured, leading to production of a fiber bundle having low porosity. On the other hand, in cases where hollow-fiber membranes with high crimping degree are used, gaps between the hollow-fiber membranes uniformly increase due to bends in the individual fibers, leading to production of a fiber bundle having high porosity. Further, in a filtration module composed of hollow-fiber membranes having low crimping degree, gaps in the fiber bundle decrease especially in cases where external pressure is applied thereto, causing increase in the flow resistance, leading to inefficient transmission of the filtration pressure to the central portion of the fiber bundle. Further, when backwashing or flushing is carried out to peel off filtration deposit from the hollow-fiber membranes, the washing effect may be smaller inside the fiber bundle. A fiber bundle composed of hollow-fiber membranes with high crimping degree has high porosity, and gaps between the hollow-fiber membranes are maintained even in cases where external pressure is applied during filtration.

The crimping degree of the hollow-fiber membrane is preferably within the range between 1.3 and 2.5. A crimping degree of less than 1.3 is not preferred because of the above-described reason, and a crimping degree of more than 2.5 may decrease the filtration area per volume, which is not preferred. In general, hollow-fiber membranes have straight tubular shapes without bends. Therefore, it is highly possible that, when they are bundled to prepare a filtration module, gaps between the hollow fibers cannot be secured, leading to production of a fiber bundle having low porosity. On the other hand, in cases where hollow-fiber membranes with high crimping degree are used, gaps between the hollow-fiber membranes uniformly increase due to bends in the individual fibers, leading to production of a fiber bundle having high porosity. Further, in a filtration module composed of hollow-fiber membranes having low crimping degree, gaps in the fiber bundle decreases especially in cases where external pressure is applied thereto, causing increase in the flow resistance, leading to inefficient transmission of the filtration pressure to the central portion of the fiber bundle. Further, when backwashing or flushing is carried out to peel off filtration deposit from the hollow-fiber membranes, the washing effect may be smaller inside the fiber bundle. A fiber bundle composed of hollow-fiber membranes with high crimping degree has high porosity, and gaps between the hollow-fiber membranes are maintained even in cases where external pressure is applied during filtration, so that uneven flow is less likely to be caused. The crimping degree can be determined by bundling about 1000 hollow-fiber membranes and measuring the circumference of the hollow-fiber membrane bundle with a PET band having a width of 4 cm while applying a tension of 1 kg to the band, followed by calculation according to the following equation:

Crimping degree=(circumference [m]/π)²/((hollow-fiber diameter [m])²×number of hollow fibers).

The inner diameter of the hollow-fiber membrane is not less than 0.4 mm in view of the resistance of the liquid that flows inside the hollow fiber tube (in-tube pressure loss), and not more than 3.0 mm in view of the area of the filling membrane per unit volume. An inner diameter of not less than 0.5 mm and not more than 1.5 mm is more preferred.

In cases where the outer diameter/inner diameter ratio of the hollow-fiber membrane is too small, the membrane has only low resistance to tension, rupture and/or compression, while in cases where the ratio is too large, the filtration performance is low since the membrane thickness is too large relative to the membrane area, which are disadvantageous. Therefore, the outer diameter/inner diameter ratio of the hollow-fiber membrane is preferably not less than 1.3 and not more than 2.3. The ratio is more preferably not less than 1.5 and not more than 2.1, still more preferably not less than 1.6 and not more than 2.0.

The porosity of the hollow-fiber membrane needs to be not less than 40% in view of the permeability, and the porosity is preferably not less than 60%. The porosity is more preferably not less than 65% and not more than 85%, still more preferably not less than 70% and not more than 80%.

The porosity can be determined according to the following equation:

Porosity [%]=100×(wet membrane weight [g]−dry membrane weight [g])/specific gravity of water [g/cm³]/(membrane volume [cm³]).

The wet membrane herein means a membrane in which pores are filled with pure water but the hollow portion does not contain pure water. More particularly, a sample membrane having a length of 10 to 20 cm is immersed in ethanol to fill the pores with ethanol, and immersion in pure water is then repeated 4 or 5 times to sufficiently substitute the inside of the pores with pure water. The hollow-fiber membrane is then held at one end, shaken well about 5 times, held at the other end, and then shaken well about 5 times again to remove water from the hollow portion. By this, the wet membrane can be obtained. The dry membrane can be obtained by drying the wet membrane, after measuring its weight, in an oven until the weight reaches a constant value at 60° C.

The membrane volume can be determined according to the following equation:

Membrane volume [cm³]=π×{(outer diameter [cm]/2)²−(inner diameter [cm]/2)²}×membrane length [cm].

In cases where the weight of a single membrane is too small and, hence, a large error occurs when its weight is measured, a plurality of membranes may be used.

In terms of the pore size of the hollow-fiber membrane, the average pore size needs to be not less than 0.001 μm and not more than 10.0 μm. The average pore size is preferably not less than 0.05 μm and not more than 1.0 μm, more preferably not less than 0.1 μm and not more than 0.5 μm. In cases where the average pore size is less than 0.001 μm, the filtration flow rate is small, which is no preferred. In cases where the average pore size is more than 10.0 μm, separation of suspended solids cannot be effectively carried out by filtration and the membrane is likely to become clogged inside with suspended solids, leading to large decrease in the filtration rate with time, which is not preferred.

The average pore size of the membrane can be determined according to the method described in ASTM: F316-86 (also referred to as the half-dry method). It should be noted that the average pore size determined by the half-dry method is that of the layer having the minimum pore size in the membrane.

Measurement of the average pore size by the half-dry method uses ethanol as the liquid to be used, and the measurement is carried out under the standard measurement conditions of 25° C. and a pressure increase rate of 0.001 MPa/second. The average pore size [μm] can be calculated according to the following equation:

Average pore size [μm]=(2860×surface tension [mN/m])/half-dry air pressure [Pa].

Since the surface tension of ethanol at 25° C. is 21.97 mN/m (“Handbook of Chemistry, Fundamental Section, 3rd Ed.”, II, p. 82, The Chemical Society of Japan ed., Maruzen Publishing, Co., Ltd., 1984), the average pore size [μm] under the standard measurement conditions can be calculated as follows:

Average pore size [μm]=62834.2/(half-dry air pressure [Pa]).

The maximum pore size of the membrane can be determined based on the pressure at which an air bubble is first generated from the membrane in the half-dry method (bubble-point method). In cases where the above-described standard measurement conditions for the half-dry method are used, the pressure at which an air bubble is first generated from the hollow-fiber membrane can be used to determine the maximum pore size [μm] according to the following equation:

Maximum pore size [μm]=62834.2/(air-bubble-generating air pressure [Pa]).

The ratio between the maximum pore size and the average pore size of the membrane is preferably less than 2.0. In cases where the ratio is not less than 2.0, there may be a problem of leakage, and the effect of backwashing may be reduced.

The three-dimensional network structure means a structure where, in the cross section of the membrane, macrovoids (large pores) do not substantially exist, while pores exist such that they three-dimensionally communicate in any direction. Presence of macrovoids in the cross section of the membrane causes decrease in the membrane strength, which is not preferred, and continuous presence of macrovoids may cause leakage. “Macrovoid” means a pore having a diameter of not less than 8 μm based on a spherical approximation. In particular, in cases where the membrane has a uniform three-dimensional network structure, the blocking pore size does not substantially change even when the surface is damaged as long as there is no penetration of the damage, which is advantageous. The cross-sectional structure of the hollow-fiber membrane obtained by the production method using an inorganic fine powder is a uniform three-dimensional network structure having no macrovoids. However, elongation of the network structure in the direction of the fibers is found because of stretching.

The pure water permeability coefficient of the porous hollow-fiber membrane needs to be not less than 0.5 m³/m²/hr and not more than 15 m³/m²/hr in view of resistance to tension, rupture and compression and of the filtration performance.

The pure water permeability coefficient can be normally measured by the following method.

A wet hollow-fiber membrane having a length of about 10 cm is prepared by immersion in ethanol followed by several times of immersion in pure water. One end of the membrane is sealed and, from the other end, an injection needle is inserted into the hollow portion. Under an environment at 25° C., pure water at 25° C. is injected from the injection needle at a pressure of 50 KPa into the hollow portion, and the amount of permeated pure water on the outer surface which is obtained as the filtrate is measured, to determine the pure water permeability coefficient according to the following equation:

Pure water permeability coefficient [m³/m²/hr]=amount of permeated water [m³]/(π×membrane diameter [m]×membrane number×effective length of membrane [m]×measurement time [hrs]).

The membrane diameter herein means the outer diameter of the membrane in cases where an external-pressure-type hollow-fiber membrane is used, or the inner diameter of the membrane in cases where an internal-pressure-type hollow-fiber membrane is used. The effective length of membrane means the net membrane length after exclusion of the length of the portion where the injection needle is inserted.

One major characteristic of the hollow-fiber membrane is that it has a low tensile elastic modulus in spite of its high tensile break strength, compressive strength and compressive elastic modulus. High tensile break strength indicates that the membrane, in the form of a module, is highly resistant to fiber breakage during filtration operation or flushing. The tensile break strength needs to be not less than 5 MPa and not more than 20 MPa. In cases where the tensile break strength is less than 5 MPa, fiber breakage occurs more frequently. In cases where the tensile break strength is more than 20 MPa, the permeability is lower. The tensile break strength is preferably not less than 7 MPa.

The tensile elongation at break needs to be not less than 30% and less than 200%, preferably not less than 50% and less than 150%. In cases where the tensile elongation at break is less than 30%, membrane breakage is more likely to occur when fibers are forcibly shaken by flushing or air scrubbing, and, in cases where the tensile elongation at break is more than 200%, resistance to rupture or compression may be low, and/or the tensile elastic modulus may be high due to a low draw ratio, which is not preferred. In cases where the step of stretching and then contracting the hollow-fiber membrane is included, the possibility of breakage at a low elongation rate is very low, so that the distribution of tensile elongation at break can be narrowed.

The critical surface tension of the hollow-fiber membrane needs to be not less than 45 mN/m and not more than 75 mN/m in view of preventing adhesion of contaminants. Although the critical surface tension of polyvinylidene fluoride itself is about 33 mN/m, it can be increased to not less than 45 mN/m by treatment in an aqueous alkaline solution. Further, since the critical surface tension of an ethylene-vinyl alcohol copolymer is not less than 70 mN/m, a polyvinylidene fluoride hollow-fiber membrane coated with an ethylene-vinyl alcohol copolymer can have a critical surface tension of not less than 70 mN/m. The value of the critical surface tension of a hollow-fiber membrane is defined as the upper limit of the surface tension of a liquid with which the hollow-fiber membrane in the dry state can be wet. The value of the critical surface tension of a hollow-fiber membrane can be measured by, for example, using standard solutions for the wetting index manufactured by Wako Pure Chemical Industries, Ltd. according to JIS K 6768. More particularly, a plurality of standard solutions having a series of different surface tensions is prepared, and each of the standard solutions is dropped onto the hollow-fiber membrane. The resulting droplet is then spread on the membrane surface. The upper limit of the surface tension value, among those of the standard solutions, at which the surface can be kept wet for not less than 2 seconds without breakage of the liquid film of the dropped standard solution can be determined as the critical surface tension.

The surface of the porous hollow-fiber membrane composed of a polyvinylidene fluoride resin may be coated with an ethylene-vinyl alcohol copolymer. The porous hollow-fiber membrane including a polyvinylidene fluoride resin, whose surface is coated with an ethylenevinyl alcohol copolymer, is preferably obtained by the step of impregnating a porous hollow-fiber membrane composed of a polyvinylidene fluoride resin with an ethylene-vinyl alcohol copolymer solution comprising an ethylene-vinyl alcohol copolymer and a solvent which is inert to polyvinylidene fluoride and dissolves the ethylene-vinyl alcohol copolymer, thereby allowing permeation of the ethylene-vinyl alcohol copolymer solution into pores inside the hollow-fiber membrane, which is followed by removal of the solvent by drying from pores existing in the portion having the thickness inside the hollow-fiber membrane. By carrying out such a step, a hollow-fiber membrane having high filtration stability can be stably produced. Since an ethylene-vinyl alcohol copolymer is a material which has high contamination resistance and high heat resistance, and which is insoluble in water, the copolymer is suitable as a material for coating a membrane.

“The surface of the porous hollow-fiber membrane is coated with an ethylene-vinyl alcohol copolymer” means that the porous hollow-fiber membrane is partially coated with an ethylene-vinyl alcohol copolymer on its specific surface(s) such as the inner surface, outer surface, and/or surfaces inside the pores; or all the surfaces of the porous hollow-fiber membrane including the inner surface, outer surface, and surfaces inside the pores are coated with an ethylene-vinyl alcohol copolymer. By applying the ethylene-vinyl alcohol copolymer coating to either a specific surface(s) or the entire surfaces, filtration membranes suitable for various fermentation liquids and bacterial, microbial and cultured cells can be provided, so that filtration membranes with which stable long-term filtration is possible can be provided.

Since the polyvinylidene fluoride hollow-fiber membrane has high strength and high compression resistance, the polyvinylidene fluoride hollow-fiber membrane can be further made to have high strength, high pressure resistance and extremely excellent contamination resistance by further coating its surface with an ethylene-vinyl alcohol copolymer. Although polyvinylidene fluoride itself is hydrophobic, alkali treatment or the like increases the wettability of the surface of the polyvinylidene fluoride hollow-fiber membrane and the surfaces of the pores inside the membrane, allowing efficient coating of the membrane with an ethylene-vinyl alcohol copolymer.

An ethylene-vinyl alcohol copolymer is a crystalline thermoplastic resin synthesized by copolymerizing ethylene and vinyl acetate and saponifying (hydrolyzing) acetic ester moieties as side chains derived from vinyl acetate, thereby converting the side chains to hydroxyl groups. The ethylene content of the ethylene-vinyl alcohol copolymer is preferably not less than 20 mol % in view of the coating efficiency, and not more than 60 mol % in view of the contamination resistance. The degree of saponification is preferably as high as possible, and more preferably not less than 80 mol % in view of the mechanical strength. Especially preferably, the copolymer is substantially completely saponified, with a degree of saponification of not less than 99 mol %. An additive(s) such as an antioxidant and/or lubricant may be added as required to the ethylenevinyl alcohol copolymer as long as these do not deteriorate the purpose of our method.

The polyvinylidene fluoride hollow-fiber membrane coated with an ethylene-vinyl alcohol copolymer has a coating amount of the ethylene-vinyl alcohol copolymer, with respect to the entire hollow-fiber membrane, of preferably not less than 0.1% by weight in view of the contamination resistance against organic substances and the like, and not more than 10% by weight in view of the permeability. The coating amount is more preferably not less than 0.5% by weight and not more than 7% by weight, still more preferably not less than 1% by weight and not more than 5% by weight. The coating is preferably performed uniformly on the internal and external surfaces of the hollow-fiber membrane and the surfaces of the fine pores in the portion having the thickness inside the fiber.

It was revealed that, by using the above membrane, operation that does not require excessive power for washing the membrane surface can be more simply carried out. In this operation, the average pore size of the fluorocarbon resin polymer separation membrane having a three-dimensional network structure is not less than 0.001 μm and not more than 10.0 μm. Within this range of the average surface pore size, the pores are less likely to be clogged with contaminants in the liquid and, hence, the permeability is less likely to decrease, so that the fluorocarbon resin polymer separation membrane can be continuously used for a longer time. Further, by setting the porosity to not less than 40% in view of the permeability and setting the critical surface tension to not less than 45 mN/m and not more than 75 mN/m, the filtration performance for the fermentation liquid increases and appropriate surface tension can be kept such that substances that cause clogging are less likely to attach to the membrane so that continuous fermentation can be carried out with small transmembrane pressure difference, the membrane is less prone to clogging and, even in cases where clogging occurred, the membrane can be more easily recovered by washing compared to cases where the operation was carried out with large transmembrane pressure difference. Therefore, stable long-term filtration can be carried out more easily.

The method for producing a chemical uses a fermentation feedstock. The fermentation feedstock is not restricted as long as it promotes the growth of the microorganisms to be cultured and allows efficient production of the fermentation product of interest.

The fermentation feedstock is preferably a normal liquid medium which comprises a carbon source(s), nitrogen source(s) and/or inorganic salt(s), and/or, as required, organic micronutrient(s) such as amino acid(s) and/or vitamin(s). Examples of the carbon source(s) include sugars such as glucose, sucrose, fructose, galactose and lactose; starch-saccharified liquid, sweet potato molasses, sugar beet molasses and high test molasses containing those sugars; organic acids such as acetic acid; alcohols such as ethanol; and glycerin. Examples of the nitrogen source(s) include ammonia gas; aqueous ammonia; ammonium salts; urea; nitric acid salts; and other organic nitrogen sources which are supplementarily used, such as oilcakes, soybean-hydrolyzed liquids, casein digests, other amino acids, vitamins, corn steep liquors, yeasts or yeast extracts, meat extracts, peptides including peptones, and cells of various fermentation microorganisms and their hydrolysates. Examples of the inorganic salt(s) which may be added as appropriate include phosphoric acid salts, magnesium salts, calcium salts, iron salts and manganese salts.

In cases where the microorganism requires particular nutrients for its growth, the nutrients may be added as preparations or natural products containing these. An anti-forming agent may also be added as required. The fermentation liquid means a liquid obtained as a result of the growth of bacterial, microbial or cultured cells in a fermentation feedstock. The composition of the fermentation feedstock to be added may be changed from the composition of the fermentation feedstock at the beginning of the culture as appropriate such that production of the chemical of interest increases.

The saccharide concentration in the fermentation liquid is preferably kept at not more than 5 g/L. The reason why the saccharide concentration in the fermentation liquid is preferably kept at not more than 5 g/L is that loss of saccharides by removal of the fermentation liquid can be minimized by this.

The microorganisms or cells are usually cultured at a pH of 3 to 8 and a temperature of 15 to 40° C., but, in cases where specific high temperature microorganisms or cells are used, the culture may alternatively be carried out at a temperature of 40 to 65° C. The pH of the fermentation liquid is usually adjusted to a predetermined value within the range of 3 to 8 with an inorganic or organic acid, alkaline substance, urea, calcium carbonate, ammonia gas or the like. When the feed rate of oxygen needs to be increased, means such as those wherein oxygen is added to the air to keep the oxygen concentration at not less than 21%; the culture is carried out under pressure; the stirring rate is increased; or the aeration rate is increased; may be employed.

Batch culture or fed-batch culture may be carried out at the initial phase of the culture to increase the microorganism concentration, followed by starting continuous culture (removal). The microorganism concentration may first be increased and the microbial cells at high concentration may be seeded thereafter, followed by carrying out continuous culture from the beginning of culture. It is possible to start supplying the fermentation feedstock liquid and removal of the culture at an appropriate timing(s). The timing of the start of supplying of the fermentation feedstock liquid and the timing of the start of removal of the culture are not necessarily the same. Supplying the fermentation feedstock liquid and removal of the culture may be carried out either continuously or intermittently.

Nutrients necessary for the growth of microbial cells may be added to the fermentation feedstock liquid to allow continuous growth of the microbial cells. The concentration of the bacterial, microbial or cultured cells in the fermentation culture medium is preferably maintained high within the range which does not cause death of the bacterial, microbial or cultured cells at a high rate due to an environment of the fermentation culture medium which is inappropriate for the growth of the bacterial, microbial or cultured cells, in view of achieving efficient production. For example, by maintaining the concentration of the bacterial, microbial or cultured cells at not less than 5 g/L in terms of dry weight, a good production efficiency can be obtained.

Bacterial, microbial or cultured cells may be removed as required from the inside of the fermenter. For example, if the concentration of the bacterial, microbial or cultured cells in the fermenter is too high, clogging of the separation membrane is likely to occur. The clogging can be prevented by removal of the cells. Further, the performance of production of the chemical may vary depending on the concentration of the bacterial, microbial or cultured cells in the fermenter, and the production performance may therefore be maintained by removing the bacterial, microbial or cultured cells using as an index the production performance.

The number of the fermenter(s) is not restricted as long as the operation of continuous culture, which is carried out while fresh microbial cells having a fermentative production capacity are grown, is a continuous culture method wherein a product is produced while the microbial cells are grown. The operation of continuous culturing is usually preferably carried out in a single fermenter in view of controlling the culture. It is also possible to use a plurality of fermenters because of the small capacity of each fermenter. In such a case, the fermentation product can be obtained at high productivity also by carrying out continuous fermentation with a plurality of fermenters connected to each other, in parallel or in series, through pipes.

The microorganisms or cultured cells which may be used in the method for producing a chemical are described below.

The bacterial, microbial or cultured cells used in the method are not restricted. Examples of the bacterial, microbial or cultured cells include yeasts commonly used in fermentation industry, such as baker's yeast; bacteria such as E. coli and coryneform bacteria; filamentous fungi; actinomycetes; animal cells and insect cells. The microorganisms or cells to be used may be those isolated from natural environments, or may be those whose properties were partially modified by mutation or genetic recombination.

The chemical produced by the method for producing a chemical is not restricted as long as the chemical is a substance produced by the microorganisms or cells in a fermentation liquid. Examples of the chemical produced by the method for producing a chemical include substances that are mass-produced in the fermentation industry, such as alcohols, organic acids and nucleic acids. Examples of the alcohols include ethanol, 1,3-propanediol, 1,4-butanediol and glycerol; examples of the organic acids include acetic acid, lactic acid, pyruvic acid, succinic acid, malic acid, itaconic acid and citric acid; examples of the nucleic acids include nucleosides such as inosine and guanosine, and nucleotides such as inosinic acid and guanylic acid; and examples of the above substances also include diamine compounds such as cadaverine. Our methods may be applied also to production of substances such as enzymes, antibiotics and recombinant proteins.

The bacterial, microbial or cultured cells which may be used in the method for producing a chemical will now be described by way of concrete examples of the chemical.

When L-lactic acid is produced by the method for producing a chemical, the bacterial, microbial or cultured cells which may be used in the production of L-lactic acid are not restricted as long as those cells are bacterial cells, microorganisms or cells that can produce L-lactic acid. Lactic acid bacteria may be preferably used as the bacterial, microbial or cultured cells which can be used for producing L-lactic acid. The lactic acid bacteria herein can be defined as prokaryotic microorganisms that produce lactic acid with a yield of not less than 50% relative to glucose consumption. Preferred examples of the lactic acid bacteria include those belonging to the genus Lactobacillus, genus Pediococcus, genus Tetragenococcus, genus Camobacterium, genus Vagococcus, genus Leuconostoc, genus Oenococcus, genus Atopobium, genus Streptococcus, genus Enterococcus, genus Lactococcus and genus Bacillus. Among these, a lactic acid bacterium with high yield of lactic acid relative to glucose consumption may be selected to be preferably used for production of lactic acid.

A lactic acid bacterium with high yield of L-lactic acid, among lactic acids, relative to glucose consumption may be selected to be preferably used for production of lactic acid. L-lactic acid is an optical isomer of lactic acid, and can be clearly distinguished from its enantiomer D-lactic acid. Examples of lactic acid bacteria with high yield of L-lactic acid relative to glucose consumption include Lactobacillus yamanashiensis, Lactobacillus animalis, Lactobacillus agilis, Lactobacillus aviaries, Lactobacillus casei, Lactobacillus delbruekii, Lactobacillus paracasei, Lactobacillus rhamnosus, Lactobacillus ruminis, Lactobacillus salivarius, Lactobacillus sharpeae, Pediococcus dextrinicus, Bacillus coagulans and Lactococcus lactis, and these may be selected to be used for production of L-lactic acid.

When L-lactic acid is produced by the method for producing a chemical, bacterial, microbial or cultured cells to which the ability to produce lactic acid was artificially given can be used. For example, bacterial, microbial or cultured cells to which an L-lactate dehydrogenase gene (which may be hereinafter referred to as L-LDH) was introduced to give or enhance their ability to produce L-lactic acid may be used. Examples of the method to give or enhance the ability to produce L-lactic acid also include known methods based on chemical mutagenesis. Preferred examples of the microorganisms include recombinant microorganisms into which L-LDH was incorporated to enhance their ability to produce L-lactic acid.

When L-lactic acid is produced by the method for producing a chemical, the host for the recombinant microorganism is preferably a prokaryotic cell such as E. coli or lactic acid bacterium, or a eukaryotic cell such as yeast. The host is especially preferably yeast. Among yeasts, those belonging to the genus Saccharomyces are preferred, and Saccharomyces cerevisiae is more preferred.

The L-LDH is not restricted as long as it encodes a protein having the activity to convert reduced nicotinamide adenine dinucleotide (NADH) and pyruvic acid to oxidized nicotinamide adenine dinucleotide (NAD+) and L-lactic acid. For example, L-LDH derived from a lactic acid bacterium with high yield of L-lactic acid relative to glucose consumption may be used. L-LDHs derived from mammals may be suitably used. Among these, L-LDHs derived from Homo sapiens or derived from frog may be used. L-LDH derived from a frog belonging to Pipidae, among frogs, is preferably used, and L-LDH derived from Xenopus laevis, among the frogs belonging to Pipidae, is more preferably used.

Examples of the human or frog-derived L-LDH include genes having genetic polymorphisms and variant genes produced by mutagenesis. The term “genetic polymorphism” means a partial change in the base sequence of a gene due to spontaneous mutation in the gene. The term “mutagenesis” means artificial introduction of a mutation into a gene. Examples of the method of mutagenesis include a method using a site-directed mutagenesis kit (Mutan-K (manufactured by TAKARA BIO INC.)) and a method using a random mutagenesis kit (BD Diversify PCR Random Mutagenesis (CLONTECH)). The human or frog-derived L-LDH may have partial deletion and/or insertion in its base sequence as long as the gene encodes a protein having the activity to convert NADH and pyruvic acid to NAD+ and L-lactic acid.

When L-lactic acid is produced by the method for producing a chemical, L-lactic acid contained in the filtrate obtained by filtration through a separation membrane may be separated and purified by a combination of known methods for concentration, distillation, crystallization and the like. Examples of the method include a method wherein the pH of the filtrate is adjusted to not more than 1, followed by extraction of L-lactic acid with diethyl ether, ethyl acetate or the like; a method wherein L-lactic acid is allowed to adsorb to an ion-exchange resin, followed by washing and then elution of the L-lactic acid; a method wherein L-lactic acid is allowed to react with an alcohol in the presence of an acid catalyst to produce an ester, which is then subjected to distillation; and a method wherein L-lactic acid is crystallized as the calcium salt or the lithium salt. Preferably, water in the filtrate may be evaporated to prepare a concentrated L-lactic acid solution, which may then be subjected to distillation. The distillation is preferably carried out while water is supplied such that the water concentration in the starting distillation liquid is kept constant. After distillation of the aqueous L-lactic acid solution, water is evaporated by heating to concentrate the solution, and purified L-lactic acid at a desired concentration can thereby be obtained. In cases where an aqueous L-lactic acid solution containing a low-boiling component(s) such as ethanol and/or acetic acid was obtained as the distillate, the low-boiling component(s) is/are removed during the process of concentration of L-lactic acid in a preferred example. After the distillation, the distillate may be subjected, as required, to removal of impurities using an ion-exchange resin and/or active carbon, and/or by chromatography separation, and, by this, L-lactic acid with higher purity can be obtained.

When D-lactic acid is produced by the method for producing a chemical, the bacterial, microbial or cultured cells which may be used for production of D-lactic acid are not restricted as long as those cells are bacterial cells, microorganisms or cells that can produce D-lactic acid. Examples of the bacterial, microbial or cultured cells which may be used for production of D-lactic acid include, in terms of wild-type strains, microorganisms such as those belonging to Lactobacillus, Bacillus and Pediococcus which have the ability to synthesize D-lactic acid.

When D-lactic acid is produced by the method for producing a chemical, it is preferred to enhance the enzyme activity of D-lactate dehydrogenase (which may be hereinafter referred to as D-LDH) in a wild-type strain. As the method for enhancing the enzyme activity, known methods by chemical mutagenesis may also be used. More preferably, a gene encoding D-lactate dehydrogenase is incorporated into a microorganism to prepare a recombinant microorganism having enhanced D-lactate dehydrogenase activity.

When D-lactic acid is produced by the method for producing a chemical, the host for the recombinant microorganism is preferably a prokaryotic cell such as E. coli or lactic acid bacterium, or a eukaryotic cell such as yeast. The host is especially preferably yeast.

When L-lactic acid is produced by the method for producing a chemical, the gene encoding D-lactate dehydrogenase is preferably derived from Lactobacillus plantarum, Pediococcus acidilactici or Bacillus laevolacticus, more preferably derived from Bacillus laevolacticus.

When D-lactic acid is produced by the method for producing a chemical, D-lactic acid contained in the filtered/separated fermentation liquid may be separated and purified by a combination of known methods for concentration, distillation, crystallization and/or the like. Examples of the method include a method wherein the pH of the filtered/separated fermentation liquid is adjusted to not more than 1, followed by extraction of D-lactic acid with diethyl ether, ethyl acetate or the like; a method wherein D-lactic acid is allowed to adsorb to an ion-exchange resin, followed by washing and then elution; a method wherein L-lactic acid is allowed to react with an alcohol in the presence of an acid catalyst to produce an ester, which is then subjected to distillation; and a method wherein D-lactic acid is crystallized as the calcium salt or the lithium salt.

When D-lactic acid is produced by the method for producing a chemical, water in the filtered/separated fermentation liquid may be evaporated to prepare a concentrated L-lactic acid solution, which may then be subjected to distillation. The distillation is preferably carried out while water is supplied such that the water concentration in the starting distillation liquid is kept constant. After distillation of the aqueous D-lactic acid solution, water is evaporated by heating to concentrate the solution, and purified D-lactic acid at a desired concentration can thereby be obtained. In cases where an aqueous D-lactic acid solution containing a low-boiling component(s) (such as ethanol and/or acetic acid) was obtained as the distillate, the low-boiling component(s) is/are removed during the process of concentration of D-lactic acid in a preferred example. After the distillation, the distillate may be subjected, as required, to removal of impurities using an ion-exchange resin and/or active carbon, and/or by chromatography separation, and, by this, D-lactic acid with higher purity can be obtained.

When ethanol is produced by the method for producing a chemical, the bacterial, microbial or cultured cells which may be used for production of ethanol are not restricted as long as those cells are bacterial cells, microorganisms or cells that can produce pyruvic acid. Examples of the bacterial, microbial or cultured cells which may be used for production of ethanol include yeasts such as those belonging to the genus Saccharomyces, genus Kluyveromyces and genus Schizosaccharomyces. Among these, Saccharomyces cerevisiae (Saccharomycescere, dsiae), Kluyveromyces lactis and Schizosaccharomyces pombe may be suitably used. Bacteria belonging to the genus Lactobacillus and genus Zymomonas may also be preferably used. Among these, Lactobacillus brevis and Zymomonas mobilis are suitably used.

The bacterial, microbial or cultured cells which may be used for production of ethanol may be bacterial, microbial or cultured cells whose ability to produce ethanol was artificially enhanced. More particularly, the bacterial, microbial or cultured cells which may be used for production of ethanol may be those whose properties were partially modified by mutation or genetic recombination. Examples of the cells whose properties were partially modified include yeasts which acquired the ability to utilize raw starch by incorporation of the glucoamylase gene of a fungus belonging to Rhizopus. Preferred examples of the method of separation/purification of ethanol contained in the filtered/separated fermentation liquid produced by the production method include purification methods by distillation and concentration/purification methods using an NF, RO membrane or zeolite separate membrane.

In cases where continuous fermentation is carried out according to the method for producing a chemical, a higher production rate per volume can be obtained compared to conventional batch fermentation, so that very efficient fermentation production is possible. The production rate in the continuous culture can be calculated according to the equation below:

Fermentation production rate (g/L/hr)=concentration of product in removed liquid (g/L)×rate of removal of fermentation liquid (L/hr)/operational liquid volume of apparatus (L).

The fermentation production rate in batch culture can be determined by dividing the amount of the product (g) upon complete consumption of the carbon source in the fermentation feedstock by the time (h) required for the consumption of the carbon source and the volume (L) of the fermentation liquid at that time.

The continuous fermentation apparatus is now described below. The continuous fermentation apparatus may be applied to production of alcohols such as ethanol, 1,3-propanediol, 1,4-butanediol and glycerol; organic acids such as acetic acid, lactic acid, pyruvic acid, succinic acid, malic acid, itaconic acid and citric acid; amino acids such as L-threonine, L-lysine, L-glutamic acid, L-tryptophan, L-isoleucine, L-glutamine, L-arginine, L-alanine, L-histidine, L-proline, L-phenylalanine, L-aspartic acid, L-tyrosine, methionine, serine, valine and leucine; nucleic acids such as inosine and guanosine; diamine compounds such as cadaverine; enzymes; antibiotics; and recombinant proteins.

The continuous fermentation apparatus is an apparatus for producing a chemical by continuous fermentation, in which a fermentation liquid obtained with bacterial, microbial or cultured cells is filtered through a separation membrane to recover the chemical from the filtrate; the unfiltered liquid is retained or refluxed in the fermentation liquid; and the fermentation feedstock is added to the fermentation liquid.

The continuous fermentation apparatus used in the method for producing a chemical is described below in detail referring to drawings.

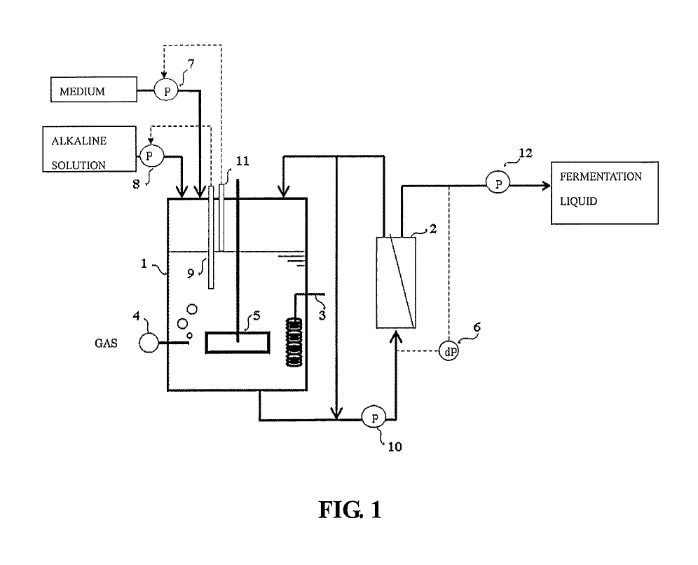

FIG. 1 is a schematic diagram for explanation of an example of the membrane-separation-type continuous fermentation apparatus used in the method for producing a chemical. In FIG. 1, the membrane-separation-type continuous fermentation apparatus is basically constituted by a fermenter 1 and a membrane separation module 2. For the membrane separation module 2, hollow-fiber membranes are used. The membrane separation module 2 is connected to the fermenter 1 through a fermentation liquid circulating pump 10.

In FIG. 1, a medium is continuously or intermittently fed to the fermenter 1 by a medium supplying pump 7. The medium may be subjected to heat disinfection or heat sterilization, or sterilization treatment using a filter before being fed, as required. Further, as required, a necessary gas may be supplied by a gas supplying device 4. The supplied gas may be recovered and recycled, followed by supplying it again with the gas supplying device 4. Further, as required, the pH of the fermentation liquid may be adjusted with a pH sensor/controlling device 9 and a pH adjustment solution supplying pump 8, and further, as required, the temperature of the fermentation liquid may be adjusted by a temperature controller 3, to carry out highly productive fermentative production.

The pH and temperature were shown as examples of physicochemical conditions of the fermentation liquid to be controlled by instrumentation/control devices, but, as required, dissolved oxygen and/or ORP may be controlled, and, by an analysis device such as an on-line chemical sensor, the concentration of the chemical in the fermentation liquid may be measured followed by controlling physicochemical conditions using as an index the concentration of the chemical in the fermentation liquid. When the continuous or intermittent feeding of a medium is carried out, the amount and the rate of feeding of the medium are appropriately controlled using as indices values of the physicochemical environment of the fermentation liquid, which values are measured by the instrumentation devices.

The fermentation liquid in the apparatus is circulated between the fermenter 1 and the separation membrane module 2 by the fermentation liquid circulating pump 10. The fermentation liquid containing the fermentation product is filtered/separated by the separation membrane module 2 into microorganisms and the fermentation product, which may then be removed from the apparatus system. When necessary, the pressure on the pipe for the fermentation liquid sent by the circulating pump and the pressure on the pipe for the filtrate obtained by filtration may be measured to determine the differential pressure, which may then be used to control the membrane filtration operation. The filtered/separated microorganisms may be allowed to remain in the apparatus system, and the microorganism concentration in the apparatus system can thereby be kept high, so that highly productive fermentation production is possible.

Further, as required, a necessary gas may be supplied into the separation membrane module 2 by the gas supplying device 4. The supplied gas may be recovered and recycled, followed by supplying it again with the gas supplying device 4. The filtration/separation with the separation membrane module 2 may be carried out, as required, by suction filtration with a filtration pump 12 or the like, or by pressurizing the inside of the apparatus system. The bacterial, microbial or cultured cells may be cultured in a culture vessel by continuous fermentation and supplied into the fermenter as required. By culturing the bacterial, microbial or cultured cells in a culture vessel by continuous fermentation and supplying these into the fermenter as required, continuous fermentation with fresh bacterial, microbial or cultured cells having a high ability to produce a chemical is always possible, so that it is possible to carry out continuous fermentation while maintaining high productivity for a long time.

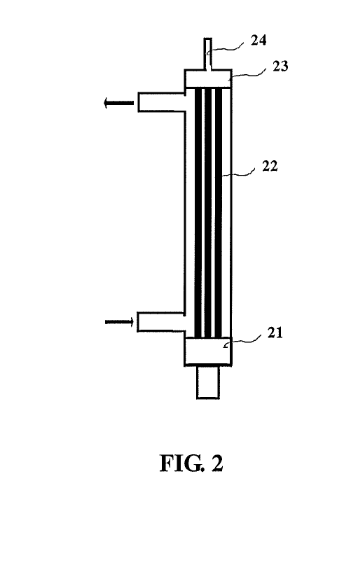

The separation membrane module shown in FIG. 2 will now be described. The separation membrane module is mainly constituted, as shown in FIG. 2, by a separation membrane bundle 22 constituted by hollow-fiber membranes, and an upper resin sealing layer 23 and a lower resin sealing layer 21. The separation membrane bundle is formed into a bundled shape by adhesion to/immobilization on the upper resin sealing layer 23 and the lower resin sealing layer 21. The hollow portions of the hollow-fiber membranes are sealed by the adhesion to/immobilization on the lower resin sealing layer, and, with such a structure, leakage of the fermentation liquid is prevented. On the other hand, the upper resin sealing layer 23 does not seal the inner pores of the hollow-fiber membranes and, with such a structure, the filtered liquid is allowed to flow into a liquid collection pipe 24. The filtered liquid obtained by filtration though the separation membrane bundle 22 passes through the hollow portions of the hollow-fiber membranes, and is removed through the liquid collection pipe 24 to the outside of the fermentation culture vessel. As the power to remove the filtered liquid, suction filtration using the hydraulic head pressure difference, pump, liquid, gas or the like, a method by pressurizing the inside of the apparatus system, or the like may be used.

The members constituting the separation membrane module of the continuous fermentation apparatus used in the method are preferably resistant to autoclaving. If the inside of the fermentation apparatus can be sterilized, the risk of contamination with unfavorable bacterial, microbial or cultured cells during continuous fermentation can be avoided, and more stable continuous fermentation is therefore possible. The members constituting the separation membrane module are preferably resistant to treatment at 121° C. for 20 minutes, which are conditions of autoclaving. Examples of the members of the separation membrane module which may be preferably selected include metals such as stainless steel and aluminum; and resins such as polyamide resins, fluorocarbon resins, polycarbonate resins, polyacetal resins, polybutylene terephthalate resins, PVDF, modified polyphenylene ether resins and polysulfone resins.

In the continuous fermentation apparatus used in the method, the membrane separation module is preferably sterilizable. If the membrane separation module is sterilizable, contamination with microorganisms can be easily avoided.

### EXAMPLES

For a more detailed description, L-lactic acid, ethanol and succinic acid were selected as examples of the chemical described above, and concrete examples of continuous fermentation by bacterial, microbial or cultured cells having the ability to produce each chemical using the apparatus shown in the schematic drawing in FIG. 1 will now be described.

The outer diameter, inner diameter, porosity, average pore size as determined by the half-dry method, maximum pore size as determined by the bubble-point method, pure water permeability coefficient, critical surface tension, crimping degree, tensile break strength and tensile elongation at break of the obtained hollow-fiber membrane were measured by the methods described above.

Further, the tensile elastic modulus, compressive elastic modulus and instantaneous anti-compression strength of the obtained hollow-fiber membrane were measured by the following method.

### Tensile Elastic Modulus

Using a tensile tester (TENSILON (registered trademark)/RTM-100) (manufactured by Toyo Baldwin), a wet hollow-fiber membrane was pulled at a chuck distance of 50 mm and a pulling rate of 200 mm/min. and, based on the load and the displacement at break, the tensile break strength and the tensile elongation at break were determined according to the equations below. The measurement was carried out in a room at a temperature of 25° C. and a relative humidity of 40 to 70%.

Tensile break strength [Pa]=load at break [N]/cross-sectional area of membrane [m²]

The cross-sectional area of membrane [m²]=π×{(outer diameter [m]/2)²−(inner diameter [m]/2)²}.

Tensile elongation at break [%]=100×displacement at break [mm]/50 [mm]

The tensile elastic modulus [Pa] was determined by determining the load at 100% displacement based on the load at 0.1% displacement and the load at 5% displacement in the above-described tensile test and dividing the load at 100% displacement by the cross-sectional area of the membrane.

### Compressive Elastic Modulus

Using a compression measuring device (AGS-H/EZtest, manufactured by Shimadzu Corporation), a 5-mm stretch of a wet hollow-fiber membrane was subjected to measurement of the compression displacement and the load in the vertical direction relative to the longitudinal direction of the fiber using a jig for compression having a width of 5 mm. The compression rate was 1 mm/min., and the load at 100% displacement, relative to the initial diameter of the hollow-fiber membrane, was determined based on the load at 0.1% displacement and the load at 5% displacement. The load at 100% displacement was normalized with the projected cross-sectional area obtained by multiplying the initial outer diameter of the hollow fiber by 5 mm, which is the hollow-fiber membrane length, to determine the compression elastic modulus. The measurement was carried out in a room at a temperature of 25° C. and a relative humidity of 40 to 70%. The compression elastic modulus in the direction of the thickness of the caterpillar belt was measured with a dry sample in the same manner.

### Instantaneous Anti-Compression Strength

A wet hollow-fiber membrane whose one end was sealed was placed in a pressure-resistant container filled with pure water at 40° C. The outer-surface side was liquid-tightly filled with pure water, and the hollow portion in the inner-surface side was kept open to the atmosphere. The water pressure was increased with the air for 15 seconds to 0.05 MPa, and filtered liquid was obtained from the outer-surface side of the hollow fiber into the inner-surface side (the external pressure method). The amount of the liquid filtered in the 15 seconds were measured, and the pressure was then further increased for 15 seconds by 0.05 MPa, followed by measuring the amount of the liquid filtered in the 15 seconds again. This cycle was continuously repeated. During the pressure increase by continuation of the cycle, the membrane was collapsed, resulting in decrease in the amount of the filtered liquid. The pressure at which the amount of the filtered liquid was maximum was defined as the instantaneous anti-compression strength [Pa].

A picture of the cross section of the polymer separation membrane was taken using a scanning electron microscope (S-800) (manufactured by Hitachi, Ltd.) at a magnification of ×10000, and, with this picture of the membrane cross section, the presence/absence of the three-dimensional network structure and the presence/absence of macrovoids having a diameter of not less than 8 μm were confirmed.

### Reference Example 1

### Preparation of Yeast Strain Having Ability to Produce L-Lactic Acid

A yeast strain having the ability to produce L-lactic acid was established as described below. A human-derived LDH gene was linked downstream of the PDC 1 promoter on the yeast genome, to establish a yeast strain having the ability to produce L-lactic acid. Polymerase chain reaction (PCR) was carried out using La-Taq (Takara Shuzo Co., Ltd.) or KOD-Plus-polymerase (Toyobo Co., Ltd.) according to the attached instructions. After culturing and collecting a human breast cancer cell line (MCF-7), total RNA was extracted therefrom using TRIZOL Reagent (Invitrogen). The obtained total RNA was used as a template for reverse transcription reaction using Super Script Choice System (Invitrogen), and cDNA was thereby synthesized. These operations were carried out according to details in the protocol attached to each product. The obtained cDNA was used as an amplification template for the subsequent PCR.

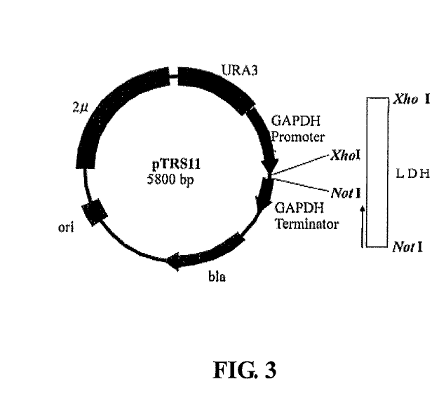

PCR was carried out using the cDNA obtained by the above-described operations as an amplification template, the oligonucleotides shown in SEQ ID NO:1 and SEQ ID NO:2 as a primer set, and KOD-Plus-polymerase, to perform cloning of the L-ldh gene. Each PCR amplification fragment was purified, and its ends were phosphorylated using T4 Polynucleotide Kinase (manufactured by TAKARA BIO INC.), followed by ligation of the fragment into pUC118 vector (prepared by digestion with HincII and dephosphorylation of the cleavage site). The ligation was carried out using DNA Ligation Kit Ver. 2 (manufactured by TAKARA BIO INC.). E. coli DH5α was transformed with the ligation plasmid product, and the plasmid DNA was recovered to obtain a plasmid in which each L-ldh gene (SEQ ID NO:3) was subcloned. The obtained pUC118 plasmid in which the L-ldh gene was inserted was digested with restriction enzymes XhoI and NotI, and each obtained DNA fragment was inserted into the XhoI/NotI cleavage site of a yeast expression vector pTRS11 (FIG. 3). Thus, a human-derived L-ldh gene expression plasmid pL-ldh5 (L-ldh gene) was obtained. The above-described pL-ldh5, which is a human-derived L-ldh gene expression vector, has been deposited, as the plasmid itself, with International Patent Organism Depositary, National Institute of Advanced Industrial Science and Technology (AIST Tsukuba Central 6, 1-1-1 Higashi, Tsukuba, Ibaraki, Japan) under the accession No. FERM AP-20421.

Using the plasmid pL-ldh5, which contains the human-derived LDH gene, as an amplification template, and the oligonucleotides represented by SEQ ID NO:4 and SEQ ID NO:5 as a primer set, PCR was carried out to amplify a 1.3-kb DNA fragment containing the human-derived LDH gene and the terminator sequence of the TDH3 gene derived from Saccharomyces cerevisiae. Further, using the plasmid pRS424 as an amplification template, and the oligonucleotides represented by SEQ ID NO:6 and SEQ ID NO:7 as a primer set, PCR was carried out to amplify a 1.2-kb DNA fragment containing the TRP1 gene derived from Saccharomyces cerevisiae. Each DNA fragment was separated by 1.5% agarose gel electrophoresis, and purified according to a conventional method.

The thus obtained 1.3-kb fragment and 1.21-kb fragment were mixed with each other. Using the resulting mixture as an amplification template, and the oligonucleotides represented by SEQ ID NO:4 and SEQ ID NO:7 as a primer set, PCR was carried out to amplify a product, which was then subjected to 1.5% agarose gel electrophoresis to prepare, according to a conventional method, a 2.5-kb DNA fragment wherein the human-derived LDH gene and the TRP1 gene are linked. The 2.5-kb DNA fragment was used for transformation of the budding yeast NBRC10505 strain into a tryptophan auxotroph.

The fact that the human-derived LDH gene is linked downstream of the PDC1 promoter in the yeast genome of the obtained transformed cells was confirmed as follows. The genomic DNA of the transformed cells was prepared according to a conventional method and, using the DNA as an amplification template and the oligonucleotides represented by SEQ ID NO:8 and SEQ ID NO:9 as a primer set, PCR was carried out to see if a 0.7-kb amplification DNA fragment is obtained thereby. Further, the fact that the transformed cells have the ability to produce lactic acid was confirmed by investigating whether the culture supernatant after culturing the transformed cells in SC medium (METHODS IN YEASTGENETICS, 2000 EDITION, CSHL PRESS) contained lactic acid, which investigation was carried out by measuring the amount of lactic acid by HPLC under the following conditions:
Column: Shim-Pack SPR—H (manufactured by Shimadzu Corporation)
Mobile phase: 5 mM p-toluenesulfonic acid (flow rate: 0.8 mL/min.)
Reaction solution: 5 mM p-toluenesulfonic acid, 20 mM Bis-Tris, 0.1 mM
EDTA.2Na (flow rate: 0.8 mL/min.)
Detection method: electric conductivity
Temperature: 45° C.

Measurement of the optical purity of L-lactic acid was carried out by HPLC under the following conditions:
Column: TSK-gel Enantio L1 (manufactured by Tosoh Corporation)
Mobile phase: 1 mM aqueous copper sulfate solution
Flow rate: 1.0 ml/min.
Detection method: UV 254 nm
Temperature: 30° C.

The optical purity of L-lactic acid was calculated by the following equation:

Optical purity (%)=100×(L−D)/(L+D)

wherein L represents the concentration of L-lactic acid, and D represents the concentration of D-lactic acid.

As a result of HPLC analysis, 4 g/L L-lactic acid was detected, and the concentration of D-lactic acid was below the detection limit. From the above study, it was confirmed that the transformants have the ability to produce L-lactic acid. The obtained transformed cells were designated the yeast SW-1 strain and used in the subsequent Examples.

### Reference Example 2

### Preparation of Porous Hollow-Fiber Membrane 1

Hydrophobic silica (manufactured by Nippon Aerosil Co., Ltd.; AEROSIL (registered trademark)-R972) having an average primary particle size of 0.016 μm and a specific surface area of 110 m²/g in an amount of 23% by weight was mixed with 30.8% by weight dioctyl phthalate and 6.2% by weight dibutyl phthalate in a Henschel mixer, and 40% by weight polyvinylidene fluoride with a weight average molecular weight of 280000 (manufactured by Kureha Chemical Industry: KF polymer #1000 (trade name)) was added thereto, followed by mixing the resulting mixture again with the Henschel mixer. The obtained mixture was further melt-kneaded with a biaxial extruder having a diameter of 48 mm, to be made into pellets.

These pellets were continuously fed to a biaxial extruder having a diameter of 30 mm, and, while the air was supplied into the hollow portion by a circular nozzle attached to the tip of the extruder, melt extrusion was carried out at 240° C. The extrudate was allowed to pass through the air over a distance of about 20 cm and then through a water bath at 40° C. at a spinning rate of 20 m/min. to be cooled and solidified, to obtain a hollow-fiber membrane. This hollow-fiber membrane was continuously retrieved by a pair of first caterpillar-belt-type retrievers at a rate of 20 m/min., and allowed to pass through a first heating vessel (0.8 m in length) whose spatial temperature was controlled to 40° C. The hollow-fiber membrane was then inserted between a pair of irregular-shaped rolls that were placed on the water surface of a cooling water bath at 20° C. and had a circumference of about 0.20 m and 4 protrusions, and the rolls were continuously operated at a rotation rate of 170 rpm to bend the membrane at constant intervals while cooling the membrane. Thereafter, the membrane was further retrieved by second caterpillar-belt-type retrievers similar to the first caterpillar-belt-type retrievers at a rate of 40 m/min., to stretch the membrane at a draw ratio of 2.0. The membrane was further allowed to pass through a second heating vessel (0.8 m in length) whose spatial temperature was controlled to 80° C., and then retrieved by third caterpillar-belt-type retrievers at a rate of 30 m/min. to achieve contraction at a ratio of 1.5, followed by being wound into a skein with a circumference of about 3 m.

Subsequently, the hollow-fiber membrane was formed into a bundle and immersed in methylene chloride at 30° C. for 1 hour, and this was repeated 5 times to extract dioctyl phthalate and dibutyl phthalate, followed by drying the membrane bundle. Thereafter, the membrane was immersed in 50% by weight aqueous ethanol solution for 30 minutes, and transferred into water and immersed therein for 30 minutes to wet the hollow-fiber membrane with water. The membrane was further immersed in 5% by weight aqueous caustic soda solution at 40° C. for 1 hour, and this was repeated twice. This was followed by 10 times of washing with water by immersing the membrane in warm water at 40° C. for 1 hour to extract hydrophobic silica, and the membrane was then dried.

The obtained hollow-fiber membrane had an average pore size of 0.29 μm as determined by the half-dry method, maximum pore size of 0.37 μm as determined by the bubble-point method, pure water permeability coefficient of 5.8 m³/m²/hr, tensile break strength of 8.5 MPa, tensile elongation at break of 135%, crimping degree of 2.45, porosity of 73% and critical surface tension of 54 mN/m. The results are summarized in Table 1.

TABLE 1
Results of measurement of hollow-fiber membrane

| | | | | | | | | | Reference | Reference | Reference |
|---|---|---|---|---|---|---|---|---|---|---|---|
| | Reference | Reference | Reference | Reference | Reference | Reference | Reference | Reference | Example | Example | Example |
| | Example 2 | Example 3 | Example 4 | Example 5 | Example 6 | Example 7 | Example 8 | Example 9 | 10 | 11 | 12 |
| Average | 0.29 | 0.15 | 0.11 | 0.90 | 0.27 | 0.13 | 0.22 | 0.20 | 1.06 | 0.22 | 0.96 |
| pore size | | | | | | | | | | | |
| (Half-dry | | | | | | | | | | | |
| method) (μm) | | | | | | | | | | | |
| Average | 0.37 | 0.24 | 0.23 | 1.22 | 0.35 | 0.24 | 0.34 | 0.28 | 1.67 | 0.32 | 1.32 |
| pore size | | | | | | | | | | | |
| (Bubble- | | | | | | | | | | | |
| point | | | | | | | | | | | |
| method) (μm) | | | | | | | | | | | |
| Pure water | 5.8 | 2.2 | 1.2 | 14.4 | 10.2 | 3.2 | 8.8 | 0.8 | 19.0 | 0.9 | 21.0 |
| permeability | | | | | | | | | | | |
| coefficient | | | | | | | | | | | |
| (m³/m²/hr) | | | | | | | | | | | |
| Breaking | 8.5 | 15.2 | 18.8 | 13.7 | 6.7 | 11.0 | 13.7 | 21.0 | 5.4 | 21.2 | 5.8 |
| strength | | | | | | | | | | | |
| (MPa) | | | | | | | | | | | |
| Elongation | 135 | 100 | 80 | 120 | 130 | 100 | 120 | 50 | 180 | 65 | 188 |
| at break (%) | | | | | | | | | | | |
| Crimping | 2.45 | 1.42 | 1.41 | 2.43 | 1.74 | 1.74 | 1.43 | 1.35 | 2.66 | 1.32 | 2.58 |
| degree | | | | | | | | | | | |
| Porosity (%) | 73 | 68 | 65 | 72 | 72 | 70 | 72 | 66 | 72 | 67 | 69 |
| Critical | 54 | 54 | 51 | 53 | 47 | 73 | 70 | 43 | 44 | 63 | 68 |
| surface | | | | | | | | | | | |
| tension | | | | | | | | | | | |
| (mN/m) | | | | | | | | | | | |
| Ethylene | No | No | No | No | No | Yes | Yes | No | No | Yes | Yes |
| vinyl | | | | | | | | | | | |
| alcohol | | | | | | | | | | | |
| coating | | | | | | | | | | | |

### Reference Example 3

### Preparation of Porous Hollow-Fiber Membrane 2

A mixture was prepared in the same manner as in Reference Example 2 except that 20% by weight hydrophobic silica and 43% by weight polyvinylidene fluoride having a weight average molecular weight of 290000 (Solef (registered trademark) 6010; manufactured by SOLVAY) were used. The obtained mixture was subjected to melting and extrusion in the same manner as in Reference Example 2, to obtain a hollow-fiber membrane.

This hollow-fiber membrane was continuously retrieved by a pair of first caterpillar-belt-type retrievers at a rate of 20 m/min., and allowed to pass through a first heating vessel (0.8 m in length) whose spatial temperature was controlled to 80° C. Thereafter, the membrane was further retrieved by second caterpillar-belt-type retrievers similar to the first caterpillar-belt-type retrievers at a rate of 40 m/min., to stretch the membrane at a draw ratio of 2. The membrane was further allowed to pass through a second heating vessel (0.8 m in length) whose spatial temperature was controlled to 80° C., and then retrieved by third caterpillar-belt-type retrievers at a rate of 30 m/min. to achieve contraction at a ratio of 1.5, followed by being wound into a skein.

The hollow-fiber membrane was then subjected to immersion in methylene chloride, immersion in 50% by weight aqueous ethanol solution, immersion in water, immersion in 5% by weight aqueous caustic soda solution and immersion in warm water in the same manner as in Reference Example 2 to extract hydrophobic silica, followed by subjecting the obtained hollow-fiber membrane to heating treatment in an oven at 140° C. for 2 hours.

The obtained hollow-fiber membrane had an average pore size of 0.15 μm as determined by the half-dry method, maximum pore size of 0.24 μm as determined by the bubble-point method, pure water permeability coefficient of 2.2 m³/m²/hr, tensile break strength of 15.2 MPa, tensile elongation at break of 100%, crimping degree of 1.42, porosity of 68% and critical surface tension of 54 mN/m. The results are summarized in Table 1.

### Reference Example 4

### Preparation of Porous Hollow-Fiber Membrane 3

A mixture was prepared using the same mixture components as in Reference Example 3 except that 18% by weight hydrophobic silica and 45% by weight of the polyvinylidene fluoride which was used in Reference Example 3 were used. The obtained mixture was subjected to melting and extrusion in the same manner as in Reference Example 3, to obtain a hollow-fiber membrane.

The obtained hollow-fiber membrane was subjected to the same treatments as in Reference Example 3 except that immersion in 20% by weight aqueous caustic soda solution was performed, to obtain a hollow-fiber membrane.

The obtained hollow-fiber membrane had an average pore size of 0.11 μm as determined by the half-dry method, maximum pore size of 0.23 μm as determined by the bubble-point method, pure water permeability coefficient of 1.2 m³/m²/hr, tensile break strength of 18.8 MPa, tensile elongation at break of 80%, crimping degree of 1.41, porosity of 65% and critical surface tension of 51 mN/m. The results are summarized in Table 1.

### Reference Example 5

### Preparation of Porous Hollow-Fiber Membrane 4

A mixture was prepared using the same mixture components as in Reference Example 3 except that 26% by weight hydrophobic silica, 33.3% by weight dioctyl phthalate, 3.7% dibutyl phthalate and 37% by weight of the polyvinylidene fluoride which was used in Reference Example 3 were used. A hollow-fiber membrane was obtained by carrying out the same steps as in Reference Example 3 except that the obtained mixture was further melt-kneaded with a biaxial extruder having a diameter of 35 mm to be made into pellets, the temperature for the melt extrusion was 230° C., and the membrane was allowed to pass through a water bath at 40° C. at a spinning rate of 20 m/min.

This hollow-fiber membrane was continuously retrieved by a pair of first caterpillar-belt-type retrievers at a rate of 10 m/min., and allowed to pass through a first heating vessel (0.8 m in length) whose spatial temperature was controlled to 40° C. Thereafter, the membrane was further retrieved by second caterpillar-belt-type retrievers similar to the first caterpillar-belt-type retrievers at a rate of 20 m/min., to stretch the membrane at a draw ratio of 2.0. The membrane was further allowed to pass through a second heating vessel (0.8 m in length) whose spatial temperature was controlled to 80° C. The hollow-fiber membrane was then inserted between a pair of irregular-shaped rolls that were placed on the water surface of a cooling water bath at 20° C. and had a circumference of about 0.20 m and 4 protrusions, and the rolls were continuously operated at a rotation rate of 170 rpm to bend the membrane at constant intervals while cooling the membrane. Thereafter, the membrane was further retrieved by third caterpillar-belt-type retrievers at a rate of 15 m/min., to achieve contraction at a ratio of 1.5, followed by being wound into a skein with a circumference of about 3 m.

The obtained hollow-fiber membrane was subjected to washing with methylene chloride, washing with ethanol, washing with water, caustic soda treatment, washing with water and drying at 140° C. in the same manner as in Reference Example 3.

The obtained hollow-fiber membrane had an average pore size of 0.90 μm as determined by the half-dry method, maximum pore size of 1.22 μm as determined by the bubble-point method, pure water permeability coefficient of 14.4 m³/m²/hr, tensile break strength of 13.7 MPa, tensile elongation at break of 120%, crimping degree of 2.43, porosity of 72% and critical surface tension of 53 mN/m. The results are summarized in Table 1.

### Reference Example 6

### Preparation of Porous Hollow-Fiber Membrane 5

A mixture was prepared in the same manner as in Reference Example 2 except that 28% by weight hydrophobic silica, 33.3% by weight dioctyl phthalate, 3.7% by weight dibutyl phthalate and 35% by weight of the polyvinylidene fluoride which was used in Reference Example 3 were used. The obtained mixture was further melt-kneaded with a biaxial extruder having a diameter of 35 mm to be made into pellets

These pellets were continuously fed to a biaxial extruder having a diameter of 30 mm, and, while the air was supplied into the hollow portion by a circular nozzle attached to the tip of the extruder, melt extrusion was carried out at 230° C. The extrudate was allowed to pass through the air over a distance of about 20 cm and then through a water bath at 40° C. at a spinning rate of 10 m/min. to be cooled and solidified, to obtain a hollow-fiber membrane.

Subsequently, the hollow-fiber membrane was formed into a bundle and immersed in methylene chloride at 30° C. for 1 hour, and this was repeated 5 times to extract dioctyl phthalate and dibutyl phthalate, followed by drying the membrane bundle. Thereafter, the hollow-fiber membrane was immersed in 50% by weight aqueous ethanol solution for 30 minutes, and transferred into water and immersed therein for 30 minutes to wet the membrane with water. The membrane was further immersed in 20% by weight aqueous caustic soda solution at 40° C. for 1 hour, and this was repeated twice. This was followed by 10 times of washing with water by immersing the membrane in warm water at 40° C. for 1 hour to extract hydrophobic silica, and the membrane was then dried.

This hollow-fiber membrane was continuously retrieved by a pair of first caterpillar-belt-type retrievers at a rate of 10 m/min., and allowed to pass through a first heating vessel (0.8 m in length) whose spatial temperature was controlled to 40° C. The hollow-fiber membrane was then inserted between a pair of irregular-shaped rolls that were placed on the water surface of a cooling water bath at 20° C. and had a circumference of about 0.20 m and 4 protrusions, and the rolls were continuously operated at a rotation rate of 170 rpm to bend the membrane at constant intervals while cooling the membrane. Thereafter, the membrane was further retrieved by second caterpillar-belt-type retrievers similar to the first caterpillar-belt-type retrievers at a rate of 20 m/min., to stretch the membrane at a draw ratio of 2.0. The membrane was further allowed to pass through a second heating vessel (0.8 m in length) whose spatial temperature was controlled to 80° C., and then retrieved by third caterpillar-belt-type retrievers at a rate of 15 m/min. to achieve contraction at a ratio of 1.5, followed by being wound into a skein with a circumference of about 3 m. The obtained hollow-fiber membrane was dried at 100° C. for 1 hour.

The obtained hollow-fiber membrane had an average pore size of 0.27 μm as determined by the half-dry method, maximum pore size of 0.35 μm as determined by the bubble-point method, pure water permeability coefficient of 10.2 m³/m²/hr, tensile break strength of 6.7 MPa, tensile elongation at break of 130%, crimping degree of 1.74, porosity of 72% and critical surface tension of 47 mN/m. The results are summarized in Table 1.

### Reference Example 7

### Preparation of Porous Hollow-Fiber Membrane 6

A mixture was prepared in the same manner as in Reference Example 3 except that 22% by weight hydrophobic silica and 41% by weight of the polyvinylidene fluoride which was used in Reference Example 3 were used. Thereafter, extrusion and spinning were carried out in the same manner as in Reference Example 3. The obtained hollow-fiber membrane was continuously retrieved by a pair of first caterpillar-belt-type retrievers at a rate of 20 m/min., and allowed to pass through a first heating vessel (0.8 m in length) whose spatial temperature was controlled to 80° C. The hollow-fiber membrane was then inserted between a pair of irregular-shaped rolls that were placed on the water surface of a cooling water bath at 20° C. and had a circumference of about 0.20 m and 4 protrusions, and the rolls were continuously operated at a rotation rate of 170 rpm to bend the membrane at constant intervals while cooling the membrane. Thereafter, the membrane was further retrieved by second caterpillar-belt-type retrievers similar to the first caterpillar-belt-type retrievers at a rate of 40 m/min., to achieve contraction at a ratio of 2.0. This was followed by stretching, contraction, washing and drying in the same manner as in Reference Example 3, to obtain a hollow-fiber membrane.

Three parts by weight of an ethylene-vinyl alcohol copolymer (Soarnol (registered trademark) ET3803; manufactured by The Nippon Synthetic Chemical Industry Co., Ltd.; ethylene content, 38 mol %) was mixed with and dissolved in 100 parts by weight of a solvent mixture comprising 50% by weight each of water and isopropyl alcohol under heat. In the obtained ethylene-vinyl alcohol copolymer solution (68° C.), a bundle composed of 100 heat-treated hollow-fiber membranes obtained each having open ends and a length of 150 cm was completely immersed for 5 minutes, and the hollow-fiber membrane bundle after being removed from the solution was dried in the air at room temperature for 30 minutes, and then dried in an oven at 60° C. for 1 hour. By this, an ethylene-vinyl-alcohol-copolymer-coated polyvinylidene fluoride hollow-fiber membrane was obtained.

The obtained hollow-fiber membrane had an average pore size of 0.13 μm as determined by the half-dry method, maximum pore size of 0.24 μm as determined by the bubble-point method, pure water permeability coefficient of 3.2 m³/m²/hr, tensile break strength of 11 MPa, tensile elongation at break of 100%, crimping degree of 1.74, porosity of 70% and critical surface tension of 73 mN/m. The results are summarized in Table 1.

### Reference Example 8

### Preparation of Porous Hollow-Fiber Membrane 7

A mixture was prepared in the same manner as in Reference Example 6 except that 25% by weight hydrophobic silica and 38% by weight of the polyvinylidene fluoride used in Reference Example 3 were used. Thereafter, the mixture was subjected to extrusion, spinning, washing with methylene chloride, washing with ethanol and washing with water in the same manner as in Reference Example 6. Subsequently, the obtained hollow-fiber membrane was continuously retrieved by a pair of first caterpillar-belt-type retrievers at a rate of 10 m/min., and allowed to pass through a first heating vessel (0.8 m in length) whose spatial temperature was controlled to 40° C. Thereafter, the membrane was further retrieved by second caterpillar-belt-type retrievers similar to the first caterpillar-belt-type retrievers at a rate of 20 m/min., to stretch the membrane at a draw ratio of 2.0. The obtained hollow-fiber membrane was then processed in the same manner as in Reference Example 6 until drying at 100° C. for 1 hour.

Using the obtained hollow-fiber membrane and an ethylene-vinyl alcohol copolymer in the same manner as in Reference Example 7, an ethylene-vinyl-alcohol-copolymer-coated polyvinylidene fluoride hollow-fiber membrane was prepared.

The obtained hollow-fiber membrane had an average pore size of 0.22 μm as determined by the half-dry method, maximum pore size of 0.34 μm as determined by the bubble-point method, pure water permeability coefficient of 8.8 m³/m²/hr, tensile break strength of 13.7 MPa, tensile elongation at break of 120%, crimping degree of 1.43, porosity of 72% and critical surface tension of 70 mN/m. The results are summarized in Table 1.

### Reference Example 9

### Preparation of Porous Hollow-Fiber Membrane 8

A mixture was prepared in the same manner as in Reference Example 2 except that 8% by weight hydrophobic silica, 33.3% by weight dioctyl phthalate, 3.7% by weight dibutyl phthalate and 55% by weight of the polyvinylidene fluoride which was used in Reference Example 3 were used. The obtained mixture was further melt-kneaded with a biaxial extruder having a diameter of 35 mm to be made into pellets.

These pellets were continuously fed to a biaxial extruder having a diameter of 30 mm and, while the air was supplied into the hollow portion by a circular nozzle attached to the tip of the extruder, melt extrusion was carried out at 230° C. The extrudate was allowed to pass through the air over a distance of about 20 cm and then through a water bath at 40° C. at a spinning rate of 10 m/min. to be cooled and solidified, to obtain a hollow-fiber membrane. This hollow-fiber membrane was continuously retrieved by a pair of first caterpillar-belt-type retrievers at a rate of 20 m/min., and allowed to pass through a first heating vessel (0.8 m in length) whose spatial temperature was controlled to 40° C., followed by being wound into a skein with a circumference of about 3 m.

Subsequently, the hollow-fiber membrane was formed into a bundle and immersed in methylene chloride at 30° C. for 1 hour, and this was repeated 5 times to extract dioctyl phthalate and dibutyl phthalate, followed by drying the membrane bundle. Thereafter, the membrane was immersed in 50% by weight aqueous ethanol solution for 30 minutes, and transferred into water and immersed therein for 30 minutes to wet the hollow-fiber membrane with water. The membrane was further immersed in 5% by weight aqueous caustic soda solution at 40° C. for 1 hour, and this was repeated twice. This was followed by 10 times of washing with water by immersing the membrane in warm water at 40° C. for 1 hour to extract hydrophobic silica, and the membrane was then dried. The obtained hollow-fiber membrane was subjected to heat treatment in an oven at 140° C. for 2 hours.

The obtained hollow-fiber membrane had an average pore size of 0.20 μm as determined by the half-dry method, maximum pore size of 0.28 μm as determined by the bubble-point method, pure water permeability coefficient of 0.8 m³/m²/hr, tensile break strength of 21 MPa, tensile elongation at break of 50%, crimping degree of 1.35, porosity of 66% and critical surface tension of 43 mN/m. The results are summarized in Table 1.

### Reference Example 10

### Preparation of Porous Hollow-Fiber Membrane 9

A mixture was prepared in the same manner as in Reference Example 5 except that 33% by weight hydrophobic silica and 30% by weight of the polyvinylidene fluoride which was used in Reference Example 3 were used. The obtained mixture was processed in the same manner as in Reference Example 9 until the step of spinning except that the spinning rate was 20 m/min. The obtained hollow-fiber membrane was continuously retrieved by a pair of first caterpillar-belt-type retrievers at a rate of 10 m/min., and allowed to pass through a first heating vessel (0.8 m in length) whose spatial temperature was controlled to 80° C., followed by being wound into a skein with a circumference of about 3 m. The obtained hollow-fiber membrane was then inserted between a pair of irregular-shaped rolls that were placed on the water surface of a cooling water bath at 20° C. and had a circumference of about 0.20 m and 4 protrusions, and the rolls were continuously operated at a rotation rate of 170 rpm to bend the membrane at constant intervals while cooling the membrane. This was repeated twice. The obtained hollow-fiber membrane was then subjected to washing with methylene chloride, washing with ethanol, caustic soda treatment, washing with water and drying in the same manner as in Reference Example 4.

The obtained hollow-fiber membrane had an average pore size of 1.06 μm as determined by the half-dry method, maximum pore size of 1.67 μm as determined by the bubble-point method, pure water permeability coefficient of 19.0 m³/m²/hr, tensile break strength of 5.4 MPa, tensile elongation at break of 180%, crimping degree of 2.66, porosity of 72% and critical surface tension of 44 mN/m. The results are summarized in Table 1.

### Reference Example 11

### Preparation of Porous Hollow-Fiber Membrane 10

Using the same mixture as in Reference Example 9, a hollow-fiber membrane was prepared and dried in the same manner, followed by using an ethylene-vinyl alcohol copolymer to prepare an ethylene-vinyl-alcohol-copolymer-coated polyvinylidene fluoride hollow-fiber membrane as in Reference Example 7.

The obtained hollow-fiber membrane had an average pore size of 0.22 μm as determined by the half-dry method, maximum pore size of 0.32 μm as determined by the bubble-point method, pure water permeability coefficient of 0.9 m³/m²/hr, tensile break strength of 21.2 MPa, tensile elongation at break of 65%, crimping degree of 1.32, porosity of 67% and critical surface tension of 63 mN/m. The results are summarized in Table 1.

### Reference Example 12

### Preparation of Porous Hollow-Fiber Membrane 11

Using the same mixture as in Reference Example 10, a hollow-fiber membrane was prepared and dried in the same manner, followed by using an ethylene-vinyl alcohol copolymer to prepare an ethylene-vinyl-alcohol-copolymer-coated polyvinylidene fluoride hollow-fiber membrane as in Reference Example 7.

The obtained hollow-fiber membrane had an average pore size of 0.96 μm as determined by the half-dry method, maximum pore size of 1.32 μm as determined by the bubble-point method, pure water permeability coefficient of 21.0 m³/m²/hr, tensile break strength of 5.8 MPa, tensile elongation at break of 188%, crimping degree of 2.58, porosity of 69% and critical surface tension of 68 mN/m. The results are summarized in Table 1.

### Example 1

### Production of L-Lactic Acid by Continuous Fermentation Using Yeast 1

Using the continuous fermentation apparatus shown in FIG. 1 and the yeast fermentation medium having the composition shown in Table 2, L-lactic acid was produced. The medium was sterilized (at 121° C. for 15 minutes) before use. As the separation membrane module member, a molded polycarbonate resin product was used. As the separation membrane, the porous hollow-fiber membrane prepared in Reference Example 2 was used. The operating conditions in Example 1 were as follows unless otherwise specified.

TABLE 2
Lactic acid fermentation medium

| Component | Amount | |
|---|---|---|
| Glucose | 100 | g |
| Yeast Nitrogen base | 6.7 | g |
| w/o amino acid (Difco) | | |
| Nineteen standard amino | 152 | mg |
| acids excluding leucine | | |
| Leucine | 760 | mg |
| Inositol | 152 | mg |
| p-Aminobenzoic acid | 16 | mg |
| Adenine | 40 | mg |
| Uracil | 152 | mg |

Unit: L⁻¹

Capacity of fermenter: 2 (L)
Capacity of membrane separation module: 0.02 (L)
Separation membrane used: polyvinylidene fluoride hollow-fiber membrane in Reference Example 2
Effective filtration area of membrane separation module: 200 (cm²)
Temperature in fermenter: 30 (° C.)
Aeration rate of fermenter: 0.05 (L/min.)
Stirring rate of fermenter: 100 (rpm)
pH adjustment: adjusted to pH 5 with 5 N NaOH
Supply of lactic acid fermentation medium: controlled based on liquid level in fermenter
Circulation rate of liquid by fermentation liquid circulator: 4 (L/min.)
Filtration flow rate: 170 mL/h (constant)

The yeast SW-1 strain established in Reference Example 1 was used as the microorganism; the lactic acid fermentation medium having the composition shown in Table 2 was used as the medium; HPLC was carried out under the conditions described in Reference Example 1 for evaluation of the concentration of lactic acid as the product; and Glucose Test Wako C (Wako Pure Chemical Industries, Ltd.) was used for measurement of the glucose concentration.

First, the SW-1 strain was cultured in 5 mL of the lactic acid fermentation medium in a test tube overnight with shaking (pre-pre-preculture). The obtained culture was inoculated in a fresh 100-mL aliquot of the lactic acid fermentation medium and subjected to culture in a 500-mL Sakaguchi flask for 24 hours at 30° C. with shaking (pre-preculture). The pre-preculture was inoculated in 1.5 L of the lactic acid fermentation medium in the continuous fermentation apparatus shown in FIG. 1, to perform culture (preculture) for 24 hours while stirring the culture in the fermenter 1 with the stirrer 5 attached thereto, controlling the aeration rate of the fermenter 1 and controlling the fermenter temperature and pH, without operating the fermentation liquid circulating pump 10. Immediately after the completion of the preculture, operation of the fermentation liquid circulating pump 10 was started, and the lactic acid fermentation medium was continuously supplied under the same operating conditions as in the preculture. L-lactic acid was produced by the continuous culture while the level of the fermentation liquid was adjusted to 2 L by controlling the liquid level in the fermenter. The concentrations of the produced L-lactic acid and the residual glucose in the liquid filtered through the membrane were measured as appropriate.

The results obtained by carrying out the continuous fermentation test for 400 hours are shown in Table 3. By our method for producing a chemical using the continuous fermentation apparatus shown in FIG. 1, stable production of L-lactic acid by continuous fermentation was possible. The transmembrane pressure difference did not exceed 10 kPa throughout the whole period of continuous fermentation.

TABLE 3
Results of lactic acid fermentation

| | Example 1 | Example 2 | Example 3 | Example 4 | Example 5 | Example 6 | Example 7 |
|---|---|---|---|---|---|---|---|
| Fermentation | 500 | 500 | 500 | 500 | 500 | 500 | 500 |
| time (hr) | | | | | | | |
| Total glucose | 5210 | 5320 | 5116 | 5118 | 5010 | 5050 | 5120 |
| fed (g) | | | | | | | |
| Total production | 3250 | 3020 | 2920 | 3110 | 3120 | 2820 | 3300 |
| of L-lactic acid (g) | | | | | | | |
| Residual | 75 | 60 | 63 | 74 | 51 | 68 | 74 |
| glucose (g) | | | | | | | |
| Yield of L-lactic | 0.62 | 0.57 | 0.57 | 0.61 | 0.62 | 0.56 | 0.64 |
| acid relative | | | | | | | |
| to glucose | | | | | | | |
| consumption (g/g) | | | | | | | |
| L-lactic acid | 3.3 | 3.0 | 2.9 | 3.1 | 3.1 | 2.8 | 3.3 |
| production | | | | | | | |
| rate (g/L/hr) | | | | | | | |

### Example 2

### Production of L-Lactic Acid by Continuous Fermentation Using Yeast 2

An L-lactic acid continuous fermentation test was carried out in the same manner as in Examples 1 using the porous hollow-fiber membrane prepared in Reference Example 3 as the separation membrane. The results are shown in Table 3. The results indicate that stable production of L-lactic acid by continuous fermentation was possible. The transmembrane pressure difference did not exceed 10 kPa throughout the whole period of continuous fermentation.

### Example 3

### Production of L-Lactic Acid by Continuous Fermentation Using Yeast 3

An L-lactic acid continuous fermentation test was carried out in the same manner as in Examples 1 using the porous hollow-fiber membrane prepared in Reference Example 4 as the separation membrane. The results are shown in Table 3. The results indicate that stable production of L-lactic acid by continuous fermentation was possible. The transmembrane pressure difference did not exceed 10 kPa throughout the whole period of continuous fermentation.

### Example 4

### Production of L-Lactic Acid by Continuous Fermentation Using Yeast 4

An L-lactic acid continuous fermentation test was carried out in the same manner as in Examples 1 using the porous hollow-fiber membrane prepared in Reference Example 5 as the separation membrane. The results are shown in Table 3. The results indicate that stable production of L-lactic acid by continuous fermentation was possible. The transmembrane pressure difference did not exceed 10 kPa throughout the whole period of continuous fermentation.

### Example 5

### Production of L-Lactic Acid by Continuous Fermentation Using Yeast 5

An L-lactic acid continuous fermentation test was carried out in the same manner as in Examples 1 using the porous hollow-fiber membrane prepared in Reference Example 6 as the separation membrane. The results are shown in Table 3. The results indicate that stable production of L-lactic acid by continuous fermentation was possible. The transmembrane pressure difference did not exceed 10 kPa throughout the whole period of continuous fermentation.

### Example 6

### Production of L-Lactic Acid by Continuous Fermentation Using Yeast 6

An L-lactic acid continuous fermentation test was carried out in the same manner as in Examples 1 using the porous hollow-fiber membrane prepared in Reference Example 7 as the separation membrane. The results are shown in Table 3. The results indicate that stable production of L-lactic acid by continuous fermentation was possible. The transmembrane pressure difference did not exceed 10 kPa throughout the whole period of continuous fermentation.

### Example 7

### Production of L-Lactic Acid by Continuous Fermentation Using Yeast 7

An L-lactic acid continuous fermentation test was carried out in the same manner as in Examples 1 using the porous hollow-fiber membrane prepared in Reference Example 8 as the separation membrane. The results are shown in Table 3. The results indicate that stable production of L-lactic acid by continuous fermentation was possible. The transmembrane pressure difference did not exceed 10 kPa throughout the whole period of continuous fermentation.

### Comparative Example 1

### Production of L-Lactic Acid by Continuous Fermentation Using Yeast 8

An L-lactic acid continuous fermentation test was carried out in the same manner as in Examples 1 using the porous hollow-fiber membrane prepared in Reference Example 9 as the separation membrane. 36 hours after the beginning of the culture, the transmembrane pressure difference exceeded 20 kPa and clogging of the membrane occurred, the continuous fermentation was stopped. Thus, it was revealed that the porous hollow-fiber membrane prepared in Reference Example 9 is not suitable for production of L-lactic acid.

### Comparative Example 2

### Production of L-Lactic Acid by Continuous Fermentation Using Yeast 9

An L-lactic acid continuous fermentation test was carried out in the same manner as in Examples 1 using the porous hollow-fiber membrane prepared in Reference Example 10 as the separation membrane. 23 hours after the beginning of the culture, the transmembrane pressure difference exceeded 20 kPa and clogging of the membrane occurred, the continuous fermentation was stopped. Thus, it was revealed that the porous hollow-fiber membrane prepared in Reference Example 10 is not suitable for production of L-lactic acid.

### Comparative Example 3

### Production of L-Lactic Acid by Continuous Fermentation Using Yeast 10

An L-lactic acid continuous fermentation test was carried out in the same manner as in Examples 1 using the porous hollow-fiber membrane prepared in Reference Example 11 as the separation membrane. 40 hours after the beginning of the culture, the transmembrane pressure difference exceeded 20 kPa and clogging of the membrane occurred, the continuous fermentation was stopped. Thus, it was revealed that the porous hollow-fiber membrane prepared in Reference Example 11 is not suitable for production of L-lactic acid.

### Comparative Example 4

### Production of L-Lactic Acid by Continuous Fermentation Using Yeast 11

An L-lactic acid continuous fermentation test was carried out in the same manner as in Examples 1 using the porous hollow-fiber membrane prepared in Reference Example 12 as the separation membrane. 28 hours after the beginning of the culture, the transmembrane pressure difference exceeded 20 kPa and clogging of the membrane occurred, the continuous fermentation was stopped. Thus, it was revealed that the porous hollow-fiber membrane prepared in Reference Example 12 is not suitable for production of L-lactic acid.

### Example 8

### Production of Ethanol by Continuous Fermentation

Using the continuous fermentation apparatus shown in FIG. 1 and the ethanol fermentation medium having the composition shown in Table 4, ethanol was produced. The medium was sterilized (at 121° C. for 15 minutes) before use. As the separation membrane, the porous hollow-fiber membrane prepared in Reference Example 4 was used. The operating conditions for the continuous fermentation test in the present Example were the same as those in Example 1 unless otherwise specified.

TABLE 4
Ethanol fermentation medium

| Component | Amount | |
|---|---|---|
| Glucose | 100 | g |
| Yeast Nitrogen base w/o amino acid (Difco) | 6.7 | g |
| Nineteen standard amino | 78 | mg |
| acids excluding leucine | | |
| Leucine | 380 | mg |
| Inositol | 76 | mg |
| p-Aminobenzoic acid | 8 | mg |
| Adenine | 40 | mg |
| Uracil | 76 | mg |

Unit: L⁻¹

Separation membrane used: polyvinylidene fluoride hollow-fiber membrane in Reference Example 4
Temperature in fermenter: 30 (° C.)

The NBRC10505 strain was used as the microorganism; the ethanol fermentation medium having the composition shown in Table 4 was used as the medium; and evaluation of the concentration of ethanol as the product was carried out by gas chromatography. The evaluation was carried out by detection/calculation using Shimadzu GC-2010 Capillary GC TC-1 (GL science) 15 meter L.*0.53 mm I.D., df 1.5 μm with a hydrogen flame ionization detector. To measure the concentration of glucose, Glucose Test Wako C (Wako Pure Chemical Industries, Ltd.) was used.

First, the NBRC10505 strain was cultured in 5 mL of the ethanol fermentation medium in a test tube overnight with shaking (pre-pre-preculture). The obtained culture was inoculated in a fresh 100-mL aliquot of the ethanol fermentation medium and subjected to culture in a 500-mL Sakaguchi flask for 24 hours at 30° C. with shaking (pre-preculture). The pre-preculture was inoculated in 1.5 L of the ethanol fermentation medium in the membrane-separation-type continuous fermentation apparatus shown in FIG. 1, to perform culture (preculture) for 24 hours while stirring the culture in the fermenter 1 with the stirrer 5 attached thereto at 100 rpm, controlling the aeration rate of the fermenter 1 and controlling the fermenter temperature and pH, without operating the fermentation liquid circulating pump 10. Immediately after the completion of the preculture, operation of the fermentation liquid circulating pump 10 was started, and the ethanol fermentation medium was continuously supplied under the same operating conditions as in the preculture. Ethanol was produced by the continuous culture while the level of the fermentation liquid was adjusted to 2 L by controlling the liquid level in the fermenter. The concentrations of the produced ethanol and the residual glucose in the liquid filtered through the membrane were measured as appropriate. The results of the measurement are shown in Table 5.

With our method using the continuous fermentation apparatus shown in FIG. 1, stable production of ethanol by continuous fermentation was possible. The transmembrane pressure difference did not exceed 10 kPa throughout the whole period of continuous fermentation.

TABLE 5
Results of ethanol fermentation

| | Example 8 |
|---|---|
| Fermentation time (hr) | 500 |
| Total glucose fed (g) | 6530 |
| Total production of ethanol (g) | 2989 |
| Residual glucose (g) | 50 |
| Yield of ethanol relative to | 0.46 |
| glucose consumption (g/g) | |
| Ethanol production rate (g/L/hr) | 5.0 |

### Example 9

### Production of Succinic Acid by Continuous Fermentation

Using the continuous fermentation apparatus shown in FIG. 1, succinic acid was produced. Succinic acid and glucose in the production of succinic acid were measured by the following methods unless otherwise specified. Succinic acid was analyzed for the centrifugation supernatant of the fermentation liquid using HPLC (Shimadzu Corporation LC10A, RI monitor: RID-10A, column: Aminex HPX-87H). The analysis was carried out at a column temperature of 50° C. The column was equilibrated with 0.01 N H₂SO₄ and the sample was then injected thereto, followed by elution with 0.01 N H₂SO₄. Glucose was measured using a glucose sensor (BF-4, manufactured by Oji Scientific Instruments). The medium was sterilized (at 121° C. for 15 minutes) before use. As the separation membrane, the porous hollow-fiber membrane prepared in Reference Example 4 was used. The operating conditions for the continuous fermentation test in this Example were the same as those in Example 1 unless otherwise specified.
Temperature in fermenter: 39 (° C.)
CO₂ flow rate in fermenter: 10 (mL/min.)
pH adjustment: adjusted to pH 6.4 with 2 M Na₂CO₃

In this Example, as a microorganism having the ability to produce succinic acid, the Anaerobiospirillum succiniciproducens ATCC53488 strain was used for continuous production of succinic acid.

In a 125-mL Erlenmeyer flask, 100 mL of a medium for seed culture comprising 20 g/L glucose, 10 g/L polypeptone, 5 g/L yeast extract, 3 g/L K₂HPO₄, 1 g/L NaCl, 1 g/L (NH₄)₂SO₄, 0.2 g/L MgCl₂ and 0.2 g/L CaCl₂.2H₂O was placed, and sterilized by heat. In an anaerobic glove box, 1 mL of 30 mM Na₂CO₃ and 0.15 mL of 180 mM H₂SO₄ were added thereto, and 0.5 mL of a reducing solution comprising 0.25 g/L cysteine.HCl and 0.25 g/L Na₂S was further added, followed by inoculation of the ATCC53488 strain and then static culture at 39° C. overnight (pre-preculture). To 1.5 L of a succinic acid fermentation medium (Table 6) placed in the continuous fermentation apparatus shown in FIG. 1, 5 mL of a reducing solution comprising 0.25 g/L cysteine.HCl and 0.25 g/L Na₂S.9H₂O was added, and 50 mL of the pre-preculture was inoculated to perform culture (preculture) for 24 hours while stirring the culture in the fermenter 1 with the stirrer 5 attached thereto at 200 rpm, controlling the CO₂ flow rate of the fermenter 1 and controlling the fermenter temperature and pH.

TABLE 6
Succinic acid fermentation medium

| Component | Amount | |
|---|---|---|
| Glucose | 50 | g |
| Polypeptone | 10 | g |
| Yeast extract | 5 | mg |
| Dipotassium | 1 | mg |
| hydrogen phosphate | | |
| Sodium chloride | 1 | mg |
| Magnesium chloride | 0.2 | mg |

Unit: L⁻¹

Immediately after the completion of the preculture, the succinic acid fermentation medium was continuously supplied, and succinic acid was produced by continuous culture while the amount of the fermentation liquid was adjusted to 2 L by controlling the liquid level in the fermenter. The concentrations of the produced succinic acid and the residual glucose in the liquid filtered through the membrane were measured as appropriate. The production rate of succinic acid and the yield of succinic acid calculated from the concentrations of succinic acid and glucose are shown in Table 7. The transmembrane pressure difference did not exceed 10 kPa throughout the whole period of continuous fermentation.

TABLE 7
Results of succinic acid fermentation

| | Example 9 |
|---|---|
| Fermentation time (hr) | 500 |
| Total glucose fed (g) | 1865 |
| Total production of succinic acid (g) | 295 |
| Residual glucose (g) | 40 |
| Yield of succinic acid relative | 0.16 |
| to glucose consumption (g/g) | |
| Succinic acid production rate (g/L/hr) | 0.3 |

### INDUSTRIAL APPLICABILITY

We provide a method for producing a chemical by continuous fermentation, wherein high productivity can be stably maintained for a long time by a simple operation method. It is thus possible to carry out continuous fermentation under simple operation conditions, wherein high productivity can be stably maintained for a long time. Therefore, chemicals as fermentation products can be stably produced at low cost generally in the fermentation industry.

1. A method of producing a chemical by continuous fermentation comprising:
filtering a fermentation liquid containing a fermentation feedstock, the chemical, and bacterial, microbial or cultured cells through a separation membrane to recover the chemical from the filtrate;
retaining or refluxing unfiltered liquid in the fermentation liquid; and
adding the fermentation feedstock to the fermentation liquid;
wherein the separation membrane is a porous hollow-fiber membrane comprising a polyvinylidene fluoride resin,
the porous hollow-fiber membrane having an average pore size of not less than 0.001 μm and not more than 10.0 μm, a pure water permeability coefficient at 50 kPa at 25° C. of not less than 0.5 m³/m²/hr and not more than 15 m³/m²/hr, a breaking strength of not less than 5 MPa and not more than 20 MPa, an elongation at break of not less than 80% and less than 1150%, a crimp amplitude of not less than 1.3 and not more than 2.5, a porosity of not less than 40%, and a critical surface tension of not less than 45 mN/m and not more than 75 mN/m.

2. The method according to claim 1, wherein a surface of the porous hollow-fiber membrane is coated with an ethylene-vinyl alcohol copolymer.

3. The method according to claim 1, wherein the porous hollow-fiber membrane is prepared by impregnating a porous hollow-fiber membrane comprising a polyvinylidene fluoride resin with an ethylene-vinyl alcohol copolymer solution comprising an ethylene-vinyl alcohol copolymer and a solvent which is inert to polyvinylidene fluoride and dissolves the ethylene-vinyl alcohol copolymer, followed by drying treatment.

4. (canceled)

5. (canceled)

6. The method according to claim 2, wherein the porous hollow-fiber membrane is prepared by impregnating a porous hollow-fiber membrane comprising a polyvinylidene fluoride resin with an ethylene-vinyl alcohol copolymer solution comprising an ethylene-vinyl alcohol copolymer and a solvent which is inert to polyvinylidene fluoride and dissolves the ethylene-vinyl alcohol copolymer, followed by drying treatment.

7. The method according to claim 1, wherein the fermentation feedstock comprises a saccharide.

8. The method according to claim 2, wherein the fermentation feedstock comprises a saccharide.

9. The method according to claim 3, wherein the fermentation feedstock comprises a saccharide.

10. The method according to claim 1, wherein the chemical is an organic acid, alcohol or nucleic acid.

11. The method according to claim 2, wherein the chemical is an organic acid, alcohol or nucleic acid.

12. The method according to claim 3, wherein the chemical is an organic acid, alcohol or nucleic acid.

13. The method according to claim 6, wherein the chemical is an organic acid, alcohol or nucleic acid.
